(19) United States
(12) Patent Application Publication
(10) Pub. No.: US 2015/0297643 A1
(43) Pub. Date: Oct. 22, 2015

(54) INHIBITION OF RHO AND OR ROCK AND CELL TRANSPLANTATION

(71) Applicant: BIOAXONE BIOSCIENCES INC., Dania Beach, FL (US)

(72) Inventor: Lisa McKerracher, Hallandale Beach, FL (US)

(21) Appl. No.: 14/418,050

(22) PCT Filed: Jul. 30, 2013
(86) PCT No.: PCT/US2013/052775

§ 371 Date: Jan. 28, 2015

Related U.S. Application Data
(60) Provisional application No. 61/678,979, filed on Aug. 2, 2012.

(51) Int. Cl. A61K 35/545; A61K 38/16; A61K 35/28; A61K 35/51; A61K 35/30; A61K 47/42; A61K 35/44

(52) U.S. Cl. CPC A61K 35/545; A61K 47/42; A61K 38/16; A61K 35/44; A61K 35/51; A61K 35/30; A61K 35/28; A61K 2035/124

(57) ABSTRACT

The present disclosure provides a multi-treatment combination to improve recovery after spinal cord injury or neurotrauma comprising: (a) Cell transplantation at the site of spinal cord injury and (b) Surgical delivery of BA-210. The combined treatment allows cells to be transplanted in the injury site during the acute trauma period, a time when the inflammatory response to neurotrauma adversely effects survival of transplanted cells. Early therapy delivered during critical care treatment after neurotrauma is essential for successful restorative therapy. The multi-treatment combination also provides a method to ensure that multi-potent transplanted cells do not become tumorigenic.

### RELATED APPLICATIONS

The present application claims priority to U.S. Provisional Application Ser. No. 61/678,979, entitled “INHIBITION OF RHO AND OR ROCK AND CELL TRANSPLANTATION”, filed Aug. 2, 2012, which is hereby incorporated by reference in its entirety.

### SEQUENCE LISTING

The present application is being filed along with a sequence listing in Electronic format. The Sequence Listing is provided as a file entitled BIOAX001WO_SEQLIST.TXT, created Jul. 29, 2013, which is approximately 3.2 kb in size. The information in the electronic format of the sequence listing is incorporated herein by reference in its entirety.

### FIELD OF THE INVENTION

The present application describes several approaches that can be used to improve recovery after spinal cord injury or neurotrauma. More specifically, embodiments include a product combination and methods of use thereof, which involve cell transplantation at the site of spinal cord injury and delivery of a RHO or ROCK inhibitor, preferably BA-210 (SEQ ID NO:1). The combined therapy allows restorative or regenerative cells to be transplanted into the injury site during the acute trauma period, a time when the inflammatory response to spinal cord injury or neurotrauma adversely effects survival of transplanted cells, and the described embodiments reduce the tumorigenic potential of implanted pluripotent cells.

### BACKGROUND OF THE INVENTION

Cell transplantation is a promising therapy for tissue regeneration and repair, particularly after neurotrauma or spinal cord injury. Cell transplantation has been identified as a treatment option for neuroinflammatory or neurodegenerative diseases that include Parkinson Disease, Alzheimer Disease, Amyotrophic Lateral Sclerosis, Multiple Sclerosis, and Huntington Disease, for example. Typically, the transplanted cells are either autologous cells, cells that are derived from the patient, or non-autologous cells, which are obtained from other sources, such as a clonal cell line.

Despite the experimental and preclinical testing of cell transplantation to treat neurotrauma and neurological disease, there are no approved therapies for human use. Part of the difficulty in reaching this goal concerns the approaches used to isolate, process, and culture the cells. Acquiring a sufficient number of cells to have therapeutic benefit and maintaining cell viability during processing has been difficult.

When non-autologous cells are used, tissue rejection poses problems for patients and the ability of some cell types to become tumorigenic, or migrate outside the tissue implantation region raises additional concerns. These issues are particularly sensitive in the context of spinal cord injury and neurotrauma because neural tumors are usually lethal.

Gliomas are a heterogeneous group of neoplasms that comprise the majority of tumors originating in the central nervous system (CNS). Malignant gliomas are so named because tumor cells phenotypically resemble normal glia even if they originated from pleuripotential precursors. In adults, the most frequently encountered gliomas are high-grade or malignant neoplasms of astrocytic and oligodendrocytic lineage, i.e., anaplastic astrocytoma (AA), glioblastoma multiforme (GBM), and anaplastic oligodendroglioma (AO), respectively. Tumors of mixed lineage are also seen, the most common of which is designated anaplastic oligoastrocytoma (AOA). In children, medulloblastomas of the cerebellum are most commonly found. The most aggressive, grade IV, astrocytoma is referred to as glioblastoma. Although cell-based therapy for spinal cord injury and neurotrauma is promising, acceptable approaches must minimize the potential to form gliomas.

The RHO signaling pathway is pivotally involved in the invasive stage of tumor cell development and metastasis (Paik J H et al. 2001, J. Biol. Chem. 276:11830-11837; Jaffe A B & Hall A. Adv. Cancer Res. 84:57-80, 2002). RHO controls cell adhesion and motility through reorganization of the actin cytoskeleton and regulation of actomyosin contractility. A number of oncogenes encode exchange factors for RHO and RHO is transcriptionally up-regulated in invasive tumors (Clark E A et al. 200. Nature 406:532-535). In neurotrauma, inflammatory mediators, such as tumor necrosis factor (TNF) cause RHO activation (Neumann et al, 2002. J. Neurosci. 22(3):854-62), further increasing the risk of uncontrolled cell growth.

While cell transplantation has been considered for the treatment of spinal cord injury, transplanted cells are typically injected long after the injury when the immediate inflammatory response resulting from the injury has abated. If cells are transplanted too early, the inflammatory environment dramatically reduces cell viability. The most urgent time to provide support to the patient, however, is immediately after spinal cord injury, neurotrauma and/or ischemia, i.e., before progressive cell death occurs, which generally takes place within the first few days after injury.

After spinal cord injury, for example, an inflammatory response takes place within minutes following the injury, and this period of inflammation last days to weeks. Most spinal cord injury patients receive surgery within the first week of injury to decompress the spinal cord, but neurosurgeons are not able to transplant cells at this time, for the reasons provided above. Therefore, all acute or subacute experimental treatments of cell transplantation to treat spinal cord injury require a second surgery to transplant the cells. The second surgery is risky for acute spinal cord injury patients, who are in a critical state and often fighting for their life.

There are many different types of cells that are being tested for cell transplantation in spinal cord injury, neurotrauma and disease (Maksymowicz (2112) Perspectives of Stem Cell-Based Therapy in Neurological Disease. Chapter 2, and Furuya et al., (2009) Brain Research 1295:192-202). Human embryonic stem cells, mesenchymal stem cells or cord blood cells have all been tested for use in neurotrauma. Certain stem cells have been isolated and characterized and cultured under conditions that induce differentiation into desired cell types. Induced pluripotent stem cells (iPS) are reprogrammed somatic cells that have potential for autologous use. For promoting regeneration in spinal cord injury, autologous olfactory ensheathing glial cells (OEGs) and Schwann cells are also under investigation. At present, it is not clear which cell lines, if any, will be beneficial to recovery of function after spinal cord injury in a human patient. The need for more approaches to improve recovery from spinal cord injury and/or neurotrauma is manifest.

### SUMMARY OF THE INVENTION

Disclosed herein are product combinations and methods for improving cell transplantation in a patient that has suffered spinal cord injury and/or neurotrauma. In some embodiments, desired cells can be transplanted at or near the site of injury during the inflammatory phase following spinal cord injury or neurotrauma allowing for an early stage therapeutic option. In some embodiments, the product combinations and methods disclosed herein, improve the viability of the transplanted cells and/or reduce the tumorigenic potential of the transplanted cells.

Methods and product combinations disclosed herein concern the use of a product combination comprising a RHO and/or a ROCK inhibitor, preferably BA-210 (SEQ ID NO:1), and a population of restorative or regenerative cells that comprise stem cells, preferably umbilical cord cells, umbilical cord blood stem cells, neural stem cells, olfactory ensheathing glial cells (OEGs), Schwann cells, embryonic stem cells, adult stem cells, bone marrow stem cells, adipose derived stem cells or restorative or regenerative cells, or iPS cells to improve recovery (e.g., axon regeneration, locomotor recovery) of a subject after spinal cord injury or neurotrauma. In some embodiments, the product combination comprising the RHO and/or ROCK inhibitor, preferably BA-210, and the restorative or regenerative cell population, preferably umbilical cord cells, umbilical cord blood stem cells, neural stem cells, olfactory ensheathing glial cells (OEGs), Schwann cells, embryonic stem cells, adult stem cells, bone marrow stem cells, adipose derived stem cells or restorative or regenerative cells, or iPS cells, are provided to a subject to reduce the invasion of monocytes that contribute to the inflammatory response; stimulate axon regeneration during the inflammatory phase following spinal cord injury or neurotrauma; enhance the survival of the transplanted restorative or regenerative cells; and/or reduce the potential for glioblastoma formation after transplantation of the restorative or regenerative cell population. In some embodiments, it is contemplated that the combination of BA-210 and a population of restorative or regenerative cells comprising stem cells, preferably umbilical cord cells, umbilical cord blood stem cells, neural stem cells, olfactory ensheathing glial cells (OEGs), Schwann cells, embryonic stem cells, adult stem cells, bone marrow stem cells, adipose derived stem cells or restorative or regenerative cells, or iPS cells, given to a subject during the inflammatory phase following spinal cord injury or neurotrauma, will synergistically improve axon regeneration, locomotor recovery, bladder control, and/or sensory perception of the subject while reducing the potential for gliobastoma formation or while reducing the incidence of gliobastoma formation.

In some embodiments, a population of restorative cells or regenerative cells that comprise stem cells are cultured with BA-210 before transplantation into a subject in need of a medicament or therapy that improves axon regeneration, locomotor recovery, bladder control, and/or sensory perception after spinal cord injury or neurotrauma. In other embodiments, BA-210 is applied directly to site of injury of the spinal cord (e.g., directly at the site of injury, caudal to the injury site or rostral to the injury site). In more embodiments, BA-210 is applied directly to site of injury of the spinal cord or at least, equal to, or any number in between 1-10 cm from the site of spinal cord injury (e.g., 1, 2, 3, 4, 5, 6, 7, 8, 9, or 10 cm) and the population of restorative cells or regenerative cells comprising stem cells, preferably umbilical cord cells, umbilical cord blood stem cells, neural stem cells, olfactory ensheathing glial cells (OEGs), Schwann cells, embryonic stem cells, adult stem cells, bone marrow stem cells, adipose derived stem cells or restorative or regenerative cells, or iPS cells, are provided before, simultaneous with, or after administration of BA-210. In some embodiments, the BA-210 and/or the aforementioned population of restorative or regenerative cells are applied to the spinal cord during the same medical procedure.

In some embodiments, a product combination that improves locomotor recovery, induces axon regeneration, improves bladder control or sensory perception after spinal cord injury (SCI) is provided. This product combination includes a population of cells comprising restorative or regenerative cells (e.g., stems cells and/or endothelial cells and/or endothelial progenitor cells), a lattice or scaffold, and an inhibitor of RHO or ROCK, such as BA-210. In some aspects of this embodiment, the lattice or scaffold includes the population of cells comprising restorative or regenerative cells.

In some aspects of this embodiment, the lattice or scaffold includes the population of cells comprising restorative or regenerative cells and an inhibitor of RHO or ROCK. In some aspects of this embodiment, the product combination may include cells that comprise restorative or regenerative cells, such as stem cells, endothelial cells, or endothelial progenitor cells. In some aspects of this embodiment the population of restorative or regenerative cells includes umbilical cord cells, umbilical cord blood stem cells, neural stem cells, olfactory ensheathing glial cells (OEGs), Schwann cells, embryonic stem cells, adult stem cells, bone marrow stem cells, adipose-derived stem cells, or iPS cells. In some aspects of this embodiment the population of restorative or regenerative cells includes uncultured restorative or regenerative cells. In some aspects of this embodiment the population of restorative or regenerative cells includes cultured restorative or regenerative cells. In some aspects of this embodiment, the population of restorative or regenerative cells includes differentiated cells. In some aspects of this embodiment, the population of restorative or regenerative cells includes undifferentiated cells. In some aspects of this embodiment the population of restorative or regenerative cells includes umbilical cord blood cell mononuclear cells (UBMC).

In some aspects of this embodiment the lattice or scaffold includes collagen, fibrin, laminin, or any combination thereof. In some aspects of this embodiment the inhibitor of RHO is BA-210. In some aspects of this embodiment the product combination and the method of use thereof optionally may include Lithium. In some aspects of this embodiment, the Lithium is formulated for oral administration (e.g., Lithium acetate) and is provided to the subject having spinal cord injury and/or neurotrauma before, during, and/or after providing the cell population that comprises restorative or regenerative cells.

In some embodiments, methods for improving locomotor recovery or inducing axon regeneration or improving bladder control or improving sensory perception in a subject after spinal cord injury (SCI) comprising providing the product combination described above to a subject in need thereof are provided. In some aspects, this method improves locomotor recovery or induces axon regeneration or improves bladder control or improves sensory perception in a subject after spinal cord injury (SCI). In some aspects, this method comprises providing an inhibitor of RHO or ROCK to a subject that has been selected for having SCI; and providing a lattice or scaffold that comprises a population of cells that comprises restorative or regenerative cells, which may further comprise stem cells, to the subject at least, equal to, or any number in between about 1, 2, 3, 4, 5, 6, 7, 8, 9, 10, 11, 12, 13, 14, 15, 16, 17, 18, 19, or 20 days after providing the inhibitor of RHO or ROCK to the subject. In some aspects of this embodiment, the population of restorative or regenerative cells comprises stem cells, endothelial cells, or endothelial progenitor cells. In some aspects of this embodiment, the population of restorative or regenerative cells comprises umbilical cord blood cells, umbilical cord blood stem cells, neural stem cells, olfactory ensheathing glial cells (OEGs), Schwann cells, embryonic stem cells, adult stem cells, bone marrow stem cells, adipose-derived stem cells, or iPS cells. In some aspects of this embodiment, the population of restorative or regenerative cells is uncultured or cultured restorative or regenerative cells. In some aspects of this embodiment, the population of restorative or regenerative cells is differentiated or undifferentiated restorative or regenerative cells. In some aspects of this embodiment, the population of cells comprises umbilical cord blood cell mononuclear cells (UBMC).

In some aspects of this embodiment, the lattice or scaffold comprises collagen, fibrin, laminin, or a combination thereof. In some aspects of this embodiment, the inhibitor of RHO or ROCK is BA-210. In some aspects of this embodiment, the lattice or scaffold is used to deliver the inhibitor of Rho or ROCK after injection of transplanted cells into the spinal cord. In some aspects of this embodiment, the method comprises providing a compound comprising Lithium (e.g., Lithium acetate) to the subject; in some aspects of the embodiment the compound comprising Lithium is formulated for oral administration. In some aspects of this embodiment, the Lithium is provided after the inhibitor of RHO or ROCK.

Additional methods that improve locomotor recovery or induce axon regeneration or improve bladder control or improve sensory perception in a subject after spinal cord injury (SCI) are provided. In some embodiments the method comprises providing a lattice or scaffold having an inhibitor of RHO or ROCK to a subject that has been selected for having SCI and providing a population of cells comprising restorative or regenerative cells, which may further comprise stem cells, to the subject at least, equal to, or any number in between about 1, 2, 3, 4, 5, 6, 7, 8, 9, 10, 11, 12, 13, 14, 15, 16, 17, 18, 19, or 20 days after providing the lattice or scaffold comprising an inhibitor of RHO or ROCK to the subject. In some aspects of this embodiment, the population of restorative or regenerative cells comprises stem cells, endothelial cells, or endothelial progenitor cells. In some aspects of this embodiment, the population of restorative or regenerative cells comprises umbilical cord blood cells, umbilical cord blood stem cells, neural stem cells, olfactory ensheathing glial cells (OEGs), Schwann cells, embryonic stem cells, adult stem cells, bone marrow stem cells, adipose derived stem cells, or iPS cells. In some aspects of this embodiment, the population of cells that comprises restorative or regenerative cells comprises uncultured or cultured restorative or regenerative cells. In some aspects of this embodiment, the population of cells that comprises restorative or regenerative cells comprises differentiated or undifferentiated restorative or regenerative cells. In some aspects of this embodiment, the population of cells that comprises restorative or regenerative cells comprises umbilical cord blood cell mononuclear cells (UBMC).

In some aspects of this embodiment, the lattice or scaffold comprises collagen, fibrin, laminin, or a combination thereof. In some aspects of this embodiment, a subject having cervical spinal cord injury is selected. In some aspects of this embodiment, a polynucleotide encoding the RHO or ROCK inhibitor is provided to the subject. In some aspects of this embodiment, the population of cells that comprises restorative or regenerative cells does not form a tumor in the subject. In some aspects of this embodiment, an additional compound comprising Lithium (e.g., Lithium acetate) is provided to the subject; in some aspects of this embodiment the compound comprising Lithium is formulated for oral administration. In some aspects the Lithium is provided after an inhibitor of RHO or ROCK.

In additional embodiments, methods of improving locomotor recovery or inducing axon regeneration or improving bladder control or improving sensory perception in a subject after spinal cord injury (SCI), which comprise providing a lattice or scaffold that comprises a population of cells that comprises restorative or regenerative cells, and an inhibitor of RHO or ROCK, to a subject that has been selected for having SCI are provided. In some embodiments the population restorative or regenerative cells does not form a tumor in said subject.

In some aspects of these embodiments, the population of restorative or regenerative cells comprises stem cells, endothelial cells, or endothelial progenitor cells. In some aspects, the population of restorative or regenerative cells comprises stem cells, endothelial cells, or endothelial progenitor cells, which may be cultured or uncultured, and may be differentiated or undifferentiated. In some aspects, the population of restorative or regenerative cells comprises umbilical cord blood cell mononuclear cells (UBMC).

In some aspects, the lattice or scaffold comprises collagen, fibrin, laminin, or any combination thereof. In some aspects, the RHO inhibitor is BA-210. In some aspects, a subject having a cervical spinal cord injury is selected. In some aspects, a polynucleotide encoding said RHO or ROCK inhibitor is provided to the subject. In some aspects, the restorative or regenerative cell population comprises a polynucleotide encoding said RHO or ROCK inhibitor. In some aspects, Lithium is provided to a subject, preferably, orally formulated Lithium. In some aspects, the Lithium is provided after the RHO or ROCK inhibitor.

Some embodiments concern methods of improving locomotor recovery or inducing axon regeneration or improving bladder control or improving sensory perception in a subject after spinal cord injury (SCI), wherein the method comprises providing an inhibitor of RHO or ROCK to a subject that has been selected for having SCI; and providing a lattice or scaffold that comprises population of cells that comprises restorative or regenerative cells to said subject. In some aspects, the inhibitor and the lattice or scaffold comprising restorative or regenerative cells are part of a first invasive treatment procedure for improving spinal cord function. In some aspects, the inhibitor of RHO and/or ROCK, lattice, and the population of cells comprising restorative or regenerative cells, which may comprise stem cells, are provided within 10 days or within 5 days of occurrence of the SCI. In some aspects, the inhibitor of RHO and/or ROCK, lattice, and the population of cells comprising restorative or regenerative cells, which may comprise stem cells, are provided before an inflammatory response of said subject has subsided or reached a point of completion. In some aspects an inflammatory response is deemed to have subsided or reached a point of completion when TNF-α levels reach a value of below about 50 pg/mL. Higher TNF-α levels generally are indicative of inflammation in SCI. In some aspects, the inflammatory response (e.g., the levels of a marker associated with inflammation, such as IL-6 or TNF-α) of the patient to receive one or more of the product combinations described herein are measured and the inflammatory response of the patient is deemed to have subsided or reached a point of completion when the levels of a marker associated with inflammation, such as TNF-α or IL-6, are at the same levels of that of a healthy person, such as a person that has not suffered spinal cord injury or neurotrauma. Stated differently, aspects of the invention include administration of one or more of the product combinations described herein to a patient that has experienced a spinal cord injury and/or a neurotrauma, wherein said product combination is provided to said patient when the TNF-α and/or IL-6 levels of said patient are above the TNF-α and/or IL-6 levels of a healthy person, such as a person that has not suffered a spinal cord injury or neurotrauma. In some aspects Lithium (e.g., Lithium acetate) and/or a lattice, such as a fibrin and/or laminin sealant, is provided to the subject. In some aspects the Lithium is formulated for oral administration. In some aspects the Lithium and/or the lattice is provided after an inhibitor of RHO or ROCK. In more embodiments, the Lithium, lattice, RHO and/or ROCK inhibitor, and the population of cells comprising restorative or regenerative cells, which may further comprise stem cells, are provided in a single composition, or in a single therapeutic method (e.g., a single surgery is conducted wherein the population of cells comprising restorative or regenerative cells, such as UBMC cells, are provided to the patient with a RHO inhibitor, such as BA-210, a fibrin and/or laminin-based sealant is applied, and the patient has been taking Lithium acetate before and after the surgery).

In some aspects the restorative or regenerative cells used in the methods described herein include stem cells, endothelial cells, or endothelial progenitor cells, which may be autologous. In some aspects, the restorative or regenerative cells include umbilical cord blood cells, umbilical cord blood stem cells, neural stem cells, olfactory ensheathing glial cells (OEGs), Schwann cells, embryonic stem cells, adult stem cells, bone marrow stem cells, adipose derived stem cells, or iPS cells. In some aspects, the cells may be cultured or uncultured, and may be differentiated or undifferentiated. In some aspects, the cells that are used in one or more of the methods described herein are umbilical cord blood cell mononuclear cells (UBMC).

In some aspects of these methods, the lattice or scaffold comprises collagen, fibrin, laminin, or a combination thereof. In some aspects, the inhibitor may be BA-210. In some aspects, a subject having a cervical spinal cord injury is selected. That is, it is contemplated that some of the product combinations described herein improve recovery from cervical spinal cord injury better than thoracic spinal cord injury, wherein the improved recovery is measured by an improved locomotor recovery or improved axon regeneration or improved bladder control or improved sensory perception. Additionally, some of the product combinations described herein improve recovery from thoracic spinal cord injury better than a treatment protocol involving administration of only restorative or regenerative cells (e.g., UBMC cells) or a RHO inhibitor (e.g., BA-210) alone, wherein the improved recovery is measured by an improved locomotor recovery or improved axon regeneration or improved bladder control or improved sensory perception.

In some of the aforementioned methods, the restorative or regenerative cells are provided to a subject having chronic spinal cord injury and these restorative or regenerative cells can comprise stem cells, endothelial cells, or endothelial progenitor cells. In some aspects, the restorative or regenerative cells used in one or more of the methods described herein comprise umbilical cord blood cells, umbilical cord blood stem cells, neural stem cells, olfactory ensheathing glial cells (OEGs), Schwann cells, embryonic stem cells, adult stem cells, bone marrow stem cells, or iPS cells. In some aspects, the restorative or regenerative cells may be cultured or uncultured, and may be differentiated or undifferentiated. In some aspects the autologous cells may comprise umbilical cord blood cell mononuclear cells (UBMC).

In some aspects the lattice or scaffold provided to a subject having chronic SCI include may comprise collagen, fibrin, laminin, or a combination thereof. In some aspects, the inhibitor may be BA-210. In some aspects, a subject having a cervical spinal cord injury is selected. In some aspects, a RHO or ROCK inhibitor is provided to the subject, and Lithium may also be provided. The Lithium can be formulated for oral administration, and in some aspects is delivered after the inhibitor of RHO or ROCK.

### BRIEF DESCRIPTION OF THE DRAWINGS

FIGS. 1A-J illustrates a comparison of the numbers of reactive mononuclear phagocytes after spinal cord injury and treatment with BA-210 (SEQ ID NO:1). The micrographs show the presence of reactive mononuclear phagocytes after SCI (FIG. 1A, FIG. 1C and FIG. 1E, FIG. 1G) and SCI+C3-07 (FIG. 1B, FIG. 1D and FIG. 1F, FIG. 1H) in rat spinal cord immunostained with ED-1 antibody. Micrographs shown in FIG. 1A, FIG. 1B, FIG. 1C, and 1D show images 4 hours and FIG. 1E, FIG. 1F, FIG. 1G, and FIG. 1H show 72 hrs after injury. Representative micrographs from grey matter (FIG. 1A, FIG. 1B, FIG. 1E, FIG. 1F) and white matter (FIG. 1C, FIG. 1D, FIG. 1G, FIG. 1H) are shown. The bar graphs show counts of ED-1 positive cells at 4 h (FIG. 1I) and 72 h (FIG. 1JJ) after injury and the results show the results of injured spinal cord alone (SCI) and injured spinal cord treated with BA-210 (SCI+BA-210) in rat spinal cord immunostained with ED-1 antibody. Counts of ED-1 positive cells in white matter and grey matter of the spinal cord at 72 h after spinal cord injury and treatment with BA-210 show reduced numbers of ED-1 positive cells in the BA-210-treated spinal cords. Cell counts were averaged from five sections 400 um apart for each of four animals per group. The asterisk indicates significant differences (p<0.05). The scale bar=50 um. C3-07 is highly related to BA-210. BA-210 was modified to make C3-07 more amenable to large scale manufacturing without changing the biological activity (Lord-Fontain et al. 2008. Local inhibition of Rho Signaling by cell permeable recombinant protein BA-210 prevents secondary damage and promotes functional recovery following acute spinal cord injury J, Neurotrauma 25:1309-1322

FIG. 2 illustrates the effect of combined myelin and inflammation on cell survival and ability of BA-210 (SEQ ID NO:1) to improve cell survival/viability in a cell culture model. The survival of PC-12 cells was studied when cells were plated on polylysine (PLL) (black bars), a growth permissive substrate, and myelin (grey bars), an inhibitory substrate. When cells are grown in the presence of TNF-α, fewer cells survive, and cells die by apoptosis. The horizontal lines shows that approximately 65% of plated cells survive when plated on myelin and exposed to TNF-α. When BA-210 is added to the cell culture media, significantly more cells survive. Bars represent mean+/−S.E.M., asterisk p≦0.05.

FIG. 3 illustrates the effect of BA-210 (SEQ ID NO:1) on a cultured glioblastoma cell line SF-268, a CNS cell line made from a human malignant glioma biopsy. In a cell proliferation assay, BA-210 exhibited cytostatic activity for this invasive cell line.

FIG. 4 illustrates the inhibition of growth of SF-268 CNS cancer cells by C3-07 measured by SRB assay. C3-07 is highly related to BA-210 (SEQ ID NO:1). BA-210 was modified to make C3-07 more amenable to large scale manufacturing without changing the biological activity (Lord-Fontain et al. 2008. Local inhibition of Rho Signaling by cell permeable recombinant protein BA-210 prevents secondary damage and promotes functional recovery following acute spinal cord injury J, Neurotrauma 25:1309-1322.

FIG. 5 illustrates the improved effect of BA-210 (SEQ ID NO:1) compared to alternate Rho inhibitors. An in vivo model was used to compare the efficacy of Rho inhibitors C3 transferase and Rho kinase inhibitors Y-27632 and BA-1049 for neuroprotection after neurotrauma. In adult rats the retinal ganglion cells were retrogradely labeled 1 week before cutting the optic nerve 1 mm from the globe Immediately after cutting the left optic nerve the test article was injected by intravitrial injection into the left eye. One week later whole mounts of the retinas were prepared from perfused animals (saline followed by paraformaldehyde). The surviving RGCs were counted under fluorescent microscopy flat mounted and the average density of retinal ganglion cells determined as RGCs/mm2 The figure show RGC survival as a percent of normal retina (normal RGCs), which is 100%. One week after axotomy, BA-210 injected in the eye completely rescues RGCs from cell death. None the Rho kinase inhibitors were as effective, and only BA-1049 at very high concentration (1 mM) had any detectable effect.

### DETAILED DESCRIPTION OF THE PREFERRED EMBODIMENT

Aspects of the present invention concern methods and product combinations related to improving locomotor recovery or inducing axon regeneration or improving bladder control or improving sensory perception in a subject after spinal cord injury (SCI) comprising a RHO inhibitor, such as BA-210 (SEQ ID NO:1), a cell population comprising a restorative or regenerative cell population, such as umbilical cord blood cell mononuclear cells (UBMC), and a lattice or framework such as one comprising fibrin and/or laminin, which may be administered to a subject suffering an SCI independent of said subject's status of inflammation (e.g., administration can take place when inflammatory markers, such as TNF-α and/or IL-6, are above the levels detected in a healthy person).

As used herein, the term “regenerative cell” refers to a cell that has the ability to give rise to (e.g., differentiate) into a specialized cell, (e.g., stem cells, endothelial cells, and/or endothelial progenitor cells). Stem cells emanate from all germinal layers (i.e., ectoderm, mesoderm, and endoderm). Typical sources of stem cells include embryos, bone marrow, peripheral blood, umbilical cord blood, placental blood, muscle tissue, and adipose tissue. Many stem cells are totipotent, meaning that they are capable of growing and differentiating into any cell in the body and some are pluripotent in that the cells are able to differentiate into a plurality of different types of specialized cells. For example, pluripotent stem cells can give rise to cells of the nervous system, skin, liver, kidney, blood, muscle, bone, etc. Examples of pluripotent stem cells include, but are not limited to, cord blood stem cells, neural stem cells, hematopoietic stem cells, adipose-derived stem cells, mesenchymal stem cells, placentally-derived stem cells, exfoliated tooth-derived stem cells, and hair follicle stem cells. In contrast, multipotent or adult stem cells typically give rise to limited types of cells.

As used herein, the term “restorative cell” refers to a cell that has the ability to provide growth factors that direct, support or restore the growth or differentiation of other cells by, for example differentiating into oligodendrocytes that remyelinate damaged axons. For example, cells that provide a permissive substrate for axon growth or that provide neurotrophic support, or both, may be considered restorative cells. In some contexts, restorative cells can also be referred to as supportive cells and restorative cells, supportive cells and/or regenerative cells can include, for example, a population of cells that comprises stem cells (e.g., a population of cells that comprises stem cells, endothelial cells, endothelial precursor cells, endothelial progenitor cells, and/or stem cell-like cells, which are capable of differentiation into one or more cell types and/or are capable of producing growth factors, induce angiogenesis, form vascular networks and/or restore the flow of blood to a tissue).

After SCI, cell transplantation is used to replace dead or damaged cells, and it is not yet known which is the ideal type of replacement cell to fill the lesion cavity that forms after contusion or impact injury. Providing living cells to replace the space in the fluid-filled cavity that forms in the spinal cord at the lesion site is beneficial to provide a cellular growth substrate for injured axons, to provide growth factors, and to replace dead cells. It is still controversial whether replacement with a single cell type is ideal, or with a regenerative cell or stem cell that can produce multiple cells types.

The term “progenitor cell” refers to cells that are lineage-committed, i.e., an individual cell can give rise to progeny limited to a single lineage. Non-limiting examples of progenitor cells include precursor cells for the neuronal, hepatic, nephrogenic, adipogenic, osteoblastic, osteoclastic, alveolar, cardiac, intestinal, or endothelial lineage.

The term “culturing” as used herein refers to contacting cells with a growth media in vitro under conditions allowing proliferation. For example, in some embodiments, stem cells are cultured in media containing BA-210 and optionally one or more growth factors, e.g., a growth factor cocktail.

The term “inhibiting apoptosis” refers to the use of a composition that delays or prevents the death of neurons and glial cells. For example, activation of RHO is associated with death of neurons and glia after neurotrauma, and the activation state of RHO can be compared in tissue or cellular homogenates of damaged or diseased tissue with normal CNS tissue.

The term “improving survival” refers to the use of a composition that increases the viability of a cell (e.g., a transplanted cell). Typically, an improvement in survival/viability and growth of a cell can be compared to a control cell population that was cultured and/or transplanted in the absence of the composition. Viable cells are cells that are alive and frequently are capable of cell division. Those of skill in the art are aware of methods to determine the viability of cells, e.g., by the ability to exclude Trypan Blue dye.

The term “reducing rejection” refers to the use of a composition to reduce, delay, or abrogate the risk of immune system rejection. Typically, the reduction in rejection of transplanted cells can be compared with the level of rejection seen with a control cell population, which was cultured and/or transplanted in the absence of the composition. As a non-limiting example, the methods of the some embodiments described herein can significantly delay the onset of immune rejection of transplanted stem cells when BA-210 is administered to a stem cell recipient.

The term “cytostatic” refers to the ability of a composition to prevent cell proliferation.

The term “BA-210” refers to C3-transferase fusion protein described in U.S. Pat. No. 7,795,218, issued Sep. 14, 2010, herein expressly incorporated by reference in its entirety. BA-210 (SEQ ID NO:1) is a novel chimeric C3-like Rho antagonist of use for promoting repair and neuron survival in injured mammalian central and peripheral nervous system and for treating or preventing cancer. At a macro level, BA-210 is most similar to C3-transferase, a secreted exoenzyme produced by Closteridium botulinum. However, in virtually every parameter analyzed, there are significant differences among these two proteins, such as physiochemical properties and in silico proteolytic peptide mapping. BA-210 lacks the N-terminal 19 amino acids of wild-type C3-transferase but retains the core catalytic domain. It is additionally modified to increase cellular permeability by addition of a novel transport sequence. It was further engineered to reduce disulfide bond-mediated aggregation and proteolysis encountered during manufacturing. Among the important properties of a therapeutic protein is its pattern of proteolytic cleavage sites. Avoidance of proteolytic cleavage during the purification process is critical, and proteolytic cleavage may alter pharmacological and/or pharmacologic properties of a therapeutic protein in the clinic. In the case of a protein purified in E. coli, the complexity increases because humans and bacteria, two organisms vastly removed in evolution are involved. The sequence of BA-210 is provided in SEQ ID NO:1.

The term “patient” refers to a mammal such as a human, needing treatment with transplanted cells to replace lost or damaged cells.

As used herein, the term “administering” refers to the delivery of a composition (e.g., restorative or regenerative cells, such as stem cells, or RHO inhibitors, such as, BA-210) by any route including, without limitation, surgical, extradural, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration. BA-210, for example, can be administered to a patient during surgery via an extradural route and optionally, the BA-210 can be provided in a fibrin matrix.

Spinal cord injury (SCI) leads to inflammatory responses such as the production of cytokines and chemokines, the activation of residential glial cells, and the infiltration of peripheral neutrophils, macrophages and T cells. The signaling molecule TNF-α is central to this response. Acceleration of these inflammatory reactions may cause the formation of glial scarring, which can be a physical barrier for regeneration, and the spread of excitotoxic products such as excitatory amino acids, free radicals and nitric oxide. Anti-inflammatory drugs such as methylprednisolone are generally used for the treatment for acute spinal cord injury.

Damage to neuronal function following SCI arises from a complex series of reactions. CNS myelin has been shown to contain molecules that are inhibitory for axonal growth. MAG, Nogo and OMgp are characterized as primary myelin proteins that inhibit the regeneration of injured spinal cord. Many of the most potent growth inhibitory proteins are concentrated in myelin and released into the CNS environment after traumatic injury transects or crushes to myelinated axons. Other inhibitory proteins such as proteoglycans are expressed by cells, which form a scar at the lesion site. Although it is not widely recognized, it is contemplated that myelin debris at or near the site of CNS trauma also contributes to poor survival or viability of transplanted cells.

RHO is a ubiquitous protein that is highly conserved from lower organisms to mammalian cells, underlying its importance in basic cell function. In neurons, RHO is an intracellular signaling protein that functions as an “on” and “off” switch to regulate the cytoskeleton and the ability of axons to regenerate. When growth inhibitory proteins interact with their neuronal receptors, RHO is turned “on” and is in the active GTP bound form. This active form blocks axon regeneration by signaling growth cone collapse. When RHO is linked to a guanidine dissociation inhibitor, a cytoplasmic protein, RHO is turned “off” signaling growth cone motility, and axons are able to regenerate, even on growth inhibitory substrates. In neurons, the RHO GTPases are activated by different receptors that are active after binding growth inhibitory proteins, such as the Nogo receptor. Nogo receptor signals to RHO and the molecular mechanism of signaling to RHO is understood.

After axonal injury in vivo, the inflammatory environment contributes substantially to the neuronal injury response. Inflammation after injury evolves with time and causes secondary damage leading to expansion of the lesion size. Expansion of the lesion results from invading inflammatory cells, which destroy injured tissue and contribute to necrosis. Studies to block these inflammatory responses show that this cascade is an important point for therapeutic intervention. Therapeutic strategies focused on this mechanism include the application of anti-integrin antibody to suppress the invasion of hematogenous macrophages (Gris, et al. (2004) J. Neurosci. 24: 16: 4043), neutralization of the Fas receptor that is expressed by injured cells in the CNS (Demjen, et al. (2004) Nat Med 10: 4: 389-95), and neutralization of the T-lymphocyte chemoattractant CXCL10 (Gonzalez, et al. (2003) Exp Neurol 184: 1: 456-63), for example.

One important pro-inflammatory cytokine released early after SCI is TNF-α, which signals the release of other pro-inflammatory cytokines (Huang, et al. (2007) Endothelium 14: 1: 25-34). Elevated levels of TNF-α appear within hours of SCI and TNF-α is expressed by activated microglia, monocytes, and reactive astrocytes. Cytokines and chemokines expressed by microglia signal invasion of hematogenous cells that secrete additional cytokines and chemokines. The presence of TNF-α in the injured CNS may block axon regeneration through a mechanism that involves the activation of the small GTPase RHO because TNF-α activates RHO in neurons expressing TNF-α receptors. Therefore, blocking RHO activation has the potential to promote regeneration by two mechanisms: blocking TNF-α signaling and blocking signaling by growth inhibitory proteins.

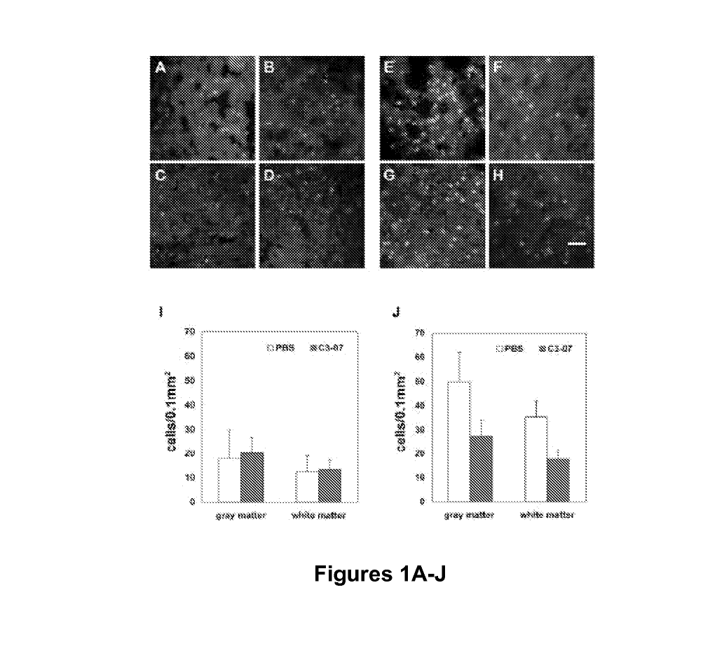

The triggering of an inflammatory response early after SCI elicits a recruitment of hematogenous macrophages into the injury site (Popovich, et al. (2003) Experimental Neurology 182: 2: 275-287). The invasion of macrophage is generally thought to be destructive and prevention of macrophage invasion can limit the extent of lesion expansion after injury and improve functional recovery. Regenerative strategies that target growth inhibitory proteins may have an impact on the macrophage migration and function, and this area of research needs more investigation (Gensel, et al. (2011) Expert Opinion on Therapeutic Targets 0: 1-14). It is reported that macrophage express the Nogo receptor, and thus blocking Nogo protein could potentially worsen the inflammatory reaction to SCI, particularly if given early after injury when the myelin inhibitors may play a role in reducing the secondary consequences of injury. By contrast, we have discovered that blocking RHO with BA-210 reduces the migration of monocytes (FIG. 1A-J). Together with the finding that cytokines can activate RHO in neurons, it has been discovered that the blocking of RHO signaling has the dual benefit of reversing RHO activation in neurons and glia (Dubreuil, et al. (2003) J. Cell Biology 162: 2: 233-43) and reducing monocyte invasion (see FIG. 1A-J).

Neuropathic pain is defined as pain initiated or caused by a primary lesion or dysfunction of the nervous system (Levendoglu et al. (2004) Gabapentin is a first line drug for the treatment of neuropathic pain in spinal cord injury. Spine, 29(7):743). Chronic pain after spinal cord injury affects up to 80% of patients, with ⅓ rating pain as severe (Cruz-Almeida et al. (2005) Chronicity of pain associated with spinal cord injury: A longitudinal analysis. Journal of rehabilitation research and development, 42(5):585). In a study of chronic SCI, 59% of patients reported onset of pain within the first six months after injury, and remission rate was only 4% within an 18 month interval (Cruz-Almeida et al. (2005) Chronicity of pain associated with spinal cord injury: A longitudinal analysis. Journal of rehabilitation research and development, 42(5):585). Other studies have reported higher pain rates (Siddall et al. (2003) A longitudinal study of the prevalence and characteristics of pain in the first 5 years following spinal cord injury. Pain, 103(3):249-57), which may be related to different types of classification of pain.

Pain following SCI presents in several ways: musculoskeletal, visceral and neuropathic. Neuropathic pain either occurs at the level of the injury (neuropathic at level pain), or occurs diffusely below the level of injury (neuropathic below level pain) (Siddall et al. (1999) Pain report and the relationship of pain to physical factors in the first 6 months following spinal cord injury. Pain, 81(1-2):187-97). Almost 40% of patients have neuropathic pain within 2 weeks of SCI (Siddall et al. (2003) A longitudinal study of the prevalence and characteristics of pain in the first 5 years following spinal cord injury. Pain, 103(3):249-57), and tetraplegics are more susceptible to below level neuropathic pain than paraplegics (Siddall et al. (2003) A longitudinal study of the prevalence and characteristics of pain in the first 5 years following spinal cord injury. Pain, 103(3):249-57).

Several studies suggest that inactivation of RHO early after SCI may reduce the burden of pain. Lysophosphatidic acid is a lipid metabolite that is released after tissue injury (Inoue et al. (2004) Initiation of neuropathic pain requires lysophosphatidic acid receptor signaling. Nature Medicine, 10(7):712-8). Lysophosphatidic acid activates RHO (Inoue et al. (2004), ibid. and Tigyi (1996) Lysophophatidic acid-induced neurite retraction in PC12 cells: neurite-protective effects of cyclic AMP signaling. J. Neurochem, 66:549-58), and Lysophosphatidic acid release may explain why injured neurons in the CNS express activated RHO (Dubreuil et al. (2006) Activation of RHO after traumatic brain injury and seizure in rats. Exp Neurol, 198(2):361-9). Treating neurons with inhibitors of RHO signaling also prevents the initiation of neuropathic pain (Inoue et al. (2004)). In one study, inhibition of RHO pathway signaling attenuated cold pain for at least 10 days (Ramer et al. (2004) RHO-kinase inhibition enhances axonal plasticity and attenuates cold hyperalgesia after dorsal rhizotomy. The Journal of neuroscience, 24(48):10796). It is not known how pain would be affected at longer time points. The continuous presence of pain can affect sleep and quality of life, and potentially could affect exercise and motor outcomes.

In patients with SCI, bladder management is of critical importance. Several levels of the spinal cord are involved in the sensory and motor control of the lower urinary tract, and SCI alters the dynamics of voiding and often requires the use of bladder drainage with catheters. Almost all patients have a neurogenic bladder, and 80% are unable to void normally (Cameron et al. (2010) Bladder management after spinal cord injury in the United States 1972 to 2005. The Journal of Urology, 184(1):213-7). The voiding dysfunction results in increased urinary tract infections (UTIs), incontinence, bladder stones, and urinary and upper tract deterioration. Preclinical experiments suggest that strategies that promote axon regeneration in the injured spinal cord also improve long term bladder function (Fouad et al. (2009) Transplantation and repair: Combined cell implantation and chondroitinase delivery prevents deterioration of bladder function in rats with complete spinal cord injury. Spinal Cord, 47(10):727-32).

After SCI, there is an initial period of spinal shock in which the bladder has no contractions and there is detrusor areflexia. Uninhibited bladder contractions gradually return after 6 to 8 weeks (Linsenmeyer T A (2002) Neurogenic bladder following spinal cord injury. Spinal Cord Medicine Philadelphia: Lippincoat Williams and Wilkins, 182-206). As uninhibited bladder contractions increase, the post voiding residua (PVR) decrease. Eventually the bladder becomes “balanced”, which the post voiding residua is less than 20% of bladder capacity in patients with detrusor hyperreflexia (Linsenmeyer T A (2002) Neurogenic bladder following spinal cord injury. Spinal Cord Medicine Philadelphia: Lippincoat Williams and Wilkins, 182-206). Some patients develop autonomic dysreflexia during voiding; one study showed 43% of patients had silent dysreflexia during voiding where blood pressure increased without other symptoms (Linsenmeyer T A (2007) Update on bladder evaluation recommendations and bladder management guideline in patients with spinal cord injury. Current Bladder Dysfunction Reports, 2(3):134-40). It is not possible to predict bladder function based on clinical neurological exam (Linsenmeyer T A (2007) Update on bladder evaluation recommendations and bladder management guideline in patients with spinal cord injury. Current Bladder Dysfunction Reports, 2(3):134-40). Therefore urodynamic evaluation is preferred to evaluate bladder function.

UTI is a common problem in SCI patients because of the uses of catheters and increased post-void residuals. At one-year after injury, 66.7 to 100% of patients have had at least one episode of UTI (Linsenmeyer T A (2002) Neurogenic bladder following spinal cord injury. Spinal Cord Medicine Philadelphia: Lippincoat Williams and Wilkins, 182-206). Pyuria, defined as more than 10 leukocytes per cubic millimeter is concurrent with UTI in 96% of patients with symptomatic infections and 46% of asymptomatic patients (Linsenmeyer TA (2002) Neurogenic bladder following spinal cord injury. Spinal Cord Medicine Philadelphia: Lippincoat Williams and Wilkins, 182-206). Pyuria may predict subsequent morbidity in SCI patients (Cardenas et al. (1995) Urinary tract infection in persons with spinal cord injury. Archives of physical medicine and rehabilitation, 76(3):272-80).

Patients with cervical spinal cord injury suffer a high incidence of autonomic dysreflexia, which is an exaggerated reflex increase in blood pressure in response to a stimulus originating below the level of injury. The most common triggers are bladder distension (often due to a blocked catheter) or bowel distension due to constipation. Dysreflexia is not always severe and can cause flushing and headaches, but the hypertension can be life threatening and can cause subarachnoid hemorrhage, seizures, and death. Autonomic dysreflexia usually develops one to three months after injury (Krassioukov et al. (2003) Autonomic dysreflexia in acute spinal cord injury: an under-recognized clinical entity. Journal of neurotrauma, 20(8):707-16) but approximately 5% of patients have an episode in the acute phase of SCI (Krassioukov et al. (2003) Autonomic dysreflexia in acute spinal cord injury: an under-recognized clinical entity. Journal of neurotrauma, 20(8):707-16); 93% of patients develop their first autonomic dysreflexic episode within one-year (Campagnolo et al. (2002) Autonomic and cardiovascular complications of spinal cord injury. Spinal Cord Medicine USA: Lippincott, Williams & Wilkins, 123-34; Weaver et al. (2002) Central mechanisms for autonomic dysreflexia after spinal cord injury. Progress in Brain Research, 137:83-95). The mechanism is still being studied, and is not known how pro-regenerative drugs may affect autonomic dysreflexia. Preclinical studies indicate that helping to re-establish serotonergic inputs into the spinal cord should prevent development of dyreflexia (Cormier et al. (2010) Development of Autonomic Dysreflexia after Spinal Cord Injury Is Associated with a Lack of Serotonergic Axons in the Intermediolateral Cell Column. Journal of neurotrauma, 27(10):1805-18) whereas ingrowth of NGF responsive fibers may increase dysreflexia (Weaver et al. (2002) Central mechanisms for autonomic dysreflexia after spinal cord injury. Progress in Brain Research, 137:83-95).

Inactivation of RHO with C3 fusion proteins or with RHO kinase inhibitors has been used to promote axon regeneration and recovery after spinal cord injury (Dubreuil et al. IBID); Dergham et al. (2002) J. Neurosci. 22: 6570-6577) and the RHO kinase inhibitor BA-210 was used in a clinical trial to treat acute spinal cord injury (Fehlings, et al. (2011) J. Neurotrauma 28: 787-796). Other investigators have tested RHO kinase inhibitors to treat spinal cord injury (Fournier, et al. (2003) J. Neurosci. 23: 4: 1416-23, Sung, et al. (2003) Brain Research 959: 1: 29-38) and targeting RHO with C3 transferase has a different effect on the spinal cord than treatment with RHO kinase inhibitors (Boomkamp S D et al. (2012) The development of a rat in vitro model of spinal cord injury demonstrating the additive effects of RHO and ROCK inhibitors on neurite outgrowth and myelination. Glia 60:441-56).

A number of RHO inhibitors are contemplated for use with a population of cells comprising restorative or regenerative cells in accordance with the methods provided herein. For example, RHO inhibitors that may be used with one or more of the population of cells comprising restorative or regenerative cells, as described herein, include Y27362, Y-39983, AR-12286, K-115 (Kowa Company, Ltd.), INS117548 (Inspire Pharmaceuticals); RKI983/SNJ-1656/Y-39983 (Novartis/Senju Pharmaceuticals/Mitsubishi Pharma Corp.), ATS907 (Altheos, Inc.), DE-104 (Santen Pharmaceuticals/Ube Industries) Thiazovivin, GSK429286A, Fasudil HCl (HA-1077), Hydroxylfasudil, (S)-(+)-2-Methyl-1-[(4-methyl-5-isoquinolinyl) sulfonyl]homopiperazine (H-1152), (S)-(+)-2-Methyl-4-glycyl-1-(4-methylisoquinolinyl-5-sulfonyl) homopiperazine, N-(4-Pyridyl)-N′-(2,4,6-trichlorophenyl) urea, 3-(4-Pyridyl)-1H-indole, N-(4-(1H-pyrazol-4-yl)phenyl)-2,3-dihydrobenzo[b][1,4]dioxine-2-carboxamide, 2,3-diaminopyrazines, benzothiophenes, N-(2-(4-Chloroanilino)-1-methyl-2-oxoethoxy)-3,5-bis(trifluoromethyl)benzamide (CCG-1423), azaindoles, azaindole 1 (6-chloro-N4-{3,5-difluoro-4-[(3-methyl-1H-pyrrolo[2,3-b]pyridin-4-yl)oxy]-phenyl}pyrimidine-2,4-diamine (Bayer), BA-1049 (U.S. Pat. No. 7,472,913 and all compounds described therein), polypeptides comprising wild-type Botulinum C-3 transferase, cell permeable C3-transferase (U.S. Pat. No. 6,855,688), polypeptides comprising functionally equivalent c-3 transferase homologues having 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or functionally equivalent c-3 transferase homologues having 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% similarity to wild-type Botulinum C-3 transferase, and polypeptides comprising BA-210. RHO activity may also be inhibited through RNAi, administration of DNA encoding a RHO inhibitor. In a preferred embodiment, the RHO inhibitor BA-210 or a nucleic acid encoding BA-210 are used. An exemplary nucleic acid encoding BA-210 is given in SEQ ID NO:2. Nucleic acids having different nucleic acid sequence, for example codon optimized for expression in a mammalian, human, yeast or bacterial system are also contemplated.

A number of inhibitors of RHO associated Protein Kinase (ROCK) are also contemplated for use with or without one or more of the aforementioned RHO inhibitors along with a population of cells comprising restorative or regenerative cells in accordance with the methods provided herein. For example, ROCK inhibitors that may be used with one or more of the population of cells comprising restorative or regenerative cells (and, optionally with one or more of the RHO inhibitors described herein), include molecules such as SAR407899, (6-chloro-N⁴-{3,5-difluoro-4-[(3-methyl-1H-pyrrolo[2,3-b]pyridin-4-yl)oxy]phenyl}pyrimidin-2,4-diamine), GSK269962A [N-(3-{[2-(4-amino-1,2,5-oxadiazol-3-yl)-1-ethyl-1H-imidazo[4,5-c]pyridin-6-yl]oxy}phenyl)-4-{[2-(4-morpholinyl)ethyl]-oxy}benzamide], and SB-7720770-B [4-(7-{[(3S)-3-amino-1-pyrrolidinyl]carbonyl}-1-ethyl-1H-imidazo[4,5-c]pyridin-2-yl)-1,2,5-oxadiazol-3-amine], as well as, other small molecule ROCK inhibitors, peptide and polypeptide inhibitors, and other molecular biological techniques such as RNAi that may reduce the total ROCK kinase activity in a cell or cells adjacent to an area suffering an SCI.

BA-210 is a biologic drug in clinical development for early hospital management of spinal cord injury (SCI). Innovative drug/therapy combinations directed at multiple and proven therapeutic targets have the potential to dramatically improve outcomes after SCI. Recognizing that certain conditions may require combination therapies, FDA issued draft guidance in 2011 for planning clinical trials with two or more investigational drugs (Woodcock et al. (2011) Development of novel combination therapies. New England Journal of Medicine, 364(11):985-7). Preclinical Proof of concept in animal models is a key requirement for approving a combination therapy for clinical trial. Accordingly, in some embodiments, it is contemplated that a product combination and methods of use thereof comprising the RHO inhibitor BA-210, and umbilical cord blood cell mononuclear cells (UCBMC) can be transplanted in a subject, preferably a human, after spinal cord injury so as to ameliorate the spinal cord injury, improve axon regeneration and locomotor recovery, improve bladder control and/or improve sensory perception of the subject. In some embodiments, Lithium, preferably an oral formulation, is also provided. In other embodiments, a lattice is also provided, preferably Haemaseel®, Tisseel® or Berplast®, or a fibrin, collagen or laminin containing sealant, as described in U.S. Pat. No. 7,141,428 and U.S. Pat. No. 7,491,692, both of which are incorporated by reference in their respective entireties.

BA-210 has pro-regenerative and neuroprotective properties. The recombinant fusion protein, BA-210 inhibits RHO, a signaling protein that controls axonal responses to growth inhibitory proteins (Fehlings et al. (2011) A phase I/IIa clinical trial of a recombinant RHO protein antagonist in acute spinal cord injury. J. Neurotrauma, 28:787-96; Lord-Fontaine et al. (2008) Local inhibition of RHO signaling by cell-permeable recombinant protein BA-210 prevents secondary damage and promotes functional recovery following acute spinal cord injury. J Neurotrauma, 25(11):1309-22). SCI activates RHO, which prevents axonal growth and causes neurons to die by apoptosis (Dubreuil et al. (2003) RHO activation patterns after spinal cord injury and the role of activated RHO in apoptosis in the central nervous system. J Cell Biol, 162(2):233-43. PMCID: 2172802). A single dose of BA-210 applied to the spinal cord injury site reverses the abnormal activation of RHO (Dubreuil et al. (2003) RHO activation patterns after spinal cord injury and the role of activated RHO in apoptosis in the central nervous system. J Cell Biol, 162(2):233-43. PMCID: 2172802). In rodent SCI models, BA-210 reduces cell death and tissue damage, promotes axon regeneration, and improves functional recovery (Lord-Fontaine et al. (2008) Local inhibition of RHO signaling by cell-permeable recombinant protein BA-210 prevents secondary damage and promotes functional recovery following acute spinal cord injury. J Neurotrauma, 25(11):1309-22; Dergham et al. (2002) RHO signaling pathway targeted to promote spinal cord repair. J Neuroscience, 22:6570-7). A multicenter phase I/IIa clinical trial has tested BA-210 in patients with SCI under regulatory guidance from FDA and Health Canada (Fehlings et al. (2011) A phase I/IIa clinical trial of a recombinant RHO protein antagonist in acute spinal cord injury. Journal of neurotrauma, 28:787-96). The drug was well tolerated and was safe at all doses tested. Treated patients with cervical injury showed functionally meaningful motor recovery.

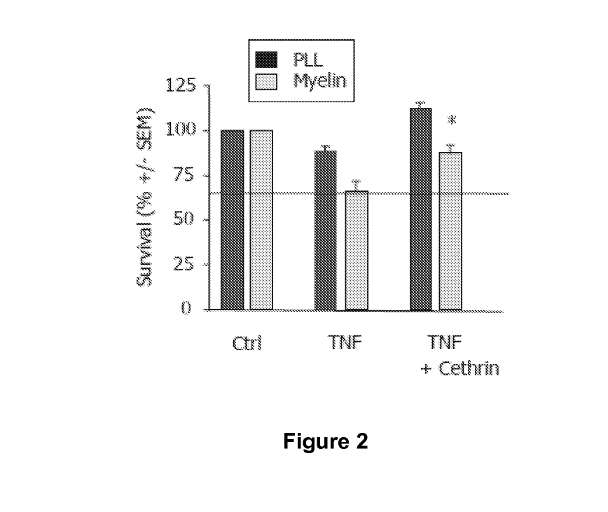

It is well established that immune cells and their inflammatory mediators are induced after CNS injury, or in neurodegenerative disease, and that chronic inflammatory responses may aggravate the degenerative processes in the CNS. It is also recognized that inflammation is a major barrier to cell transplantation strategies after CNS injury. However, it has not been recognized that myelin debris contributes to failed survival of cell transplants, and that by neutralizing the cellular response to myelin debris, cell survival/viability after transplantation can be improved. In FIG. 2, it is indicated that the combined presence of TNF-α and myelin induces neuronal cell death. The use of the RHO inhibitor BA-210 significantly increased cell survival in the presence of myelin and TNF-α, which will promote engraftment and long-term viability of cells transplanted at the site of spinal cord injury.

It is contemplated that a combination therapy is needed to improve recovery after SCI. Transplanted cells are typically injected into injured spinal cord rostral or caudal to the injury site and at 10 or more days after injury so as to avoid the inflammatory molecules that reduce the viability of the transplanted cells. By administering a RHO and/or ROCK inhibitor at or near the site of spinal cord injury, e.g., BA-210, in combination or shortly after introducing a population of restorative or regenerative cells that comprises stem cells at or near the site of spinal cord injury, e.g., umbilical cord blood cell mononuclear cells (UBMC), it is contemplated that the subject that receives the product combination will experience improved axon regeneration, improved locomotor recovery, improved bladder control and/or improved sensory perception as compared to a subject that receives a RHO and/or ROCK inhibitor at or near the site of spinal cord injury, e.g., BA-210, in the absence of a population of restorative or regenerative cells that comprises stem cells or a subject that receives a population of restorative or regenerative cells that comprises stem cells in the absence of a RHO and/or ROCK inhibitor at or near the site of spinal cord injury, e.g., BA-210. It is also contemplated that a subject having a spinal cord injury can be provided a product combination comprising a RHO and/or ROCK inhibitor at or near the site of spinal cord injury, e.g., BA-210, in combination or shortly after introducing a population of restorative or regenerative cells that comprises stem cells at or near the site of spinal cord injury, e.g., umbilical cord blood cell mononuclear cells (UBMC), in less than or equal to about 10 days (e.g., less than or equal to 1, 2, 3, 4, 5, 6, 7, 8, or 9 days) after the spinal cord injury and said subject will experience improved axon regeneration, improved locomotor recovery, improved bladder control and/or improved sensory perception; whereas, a subject having spinal cord injury that receives either the RHO and/or ROCK inhibitor (e.g., BA-210) or the population of restorative or regenerative cells that comprises stem cells separately (e.g., the cells and the inhibitor are not co-administered or not provided within 1, 2, 3, 4, 5, 6, 7, or 8 hours of one another) or only receives the RHO and/or ROCK inhibitor (e.g., BA-210) or the population of restorative or regenerative cells comprising stem cells in less than or equal to about 10 days (e.g., less than or equal to 1, 2, 3, 4, 5, 6, 7, 8, or 9 days) will not experience improved axon regeneration, improved locomotor recovery, improved bladder control and/or improved sensory perception.

Different strategies have been used to combine drug treatment with cell transplantation to increase the survival and effectiveness of transplanted cells. For example Lithium has been used to promote growth factor production of cord blood cells transplanted into damaged regions of the spinal cord (Sun and Young, U.S. Publication No. 2010/0189696). It is known that inhibition of RHO signaling can be neuroprotective when applied to injured spinal cord (Dubreuil et al, ibid). Others have extended this finding to test the ability of RHO inhibition to reduce apoptosis during transplantation. For example, the dissociation of neural precursors activated RHO and treatment with C3 transferase of RHO kinase inhibition can reduce apoptosis of stem cells when treated with RHO kinase inhibitor before transplantation. (Koyanagi, et al. (2008) Journal of Neuroscience Research 86: 2: 270-280). Another study showed that RHO kinase inhibition has protective ability for human embryonic stem cells and their ability to differentiate into neural progenitors (Watanabe, et al. (2007) Nature biotechnology 25:681-686). These studies have focused on the ability of RHO kinase pretreatment to enhance survival of cells during the dissociation process. However, these investigators did not address the ability of RHO inhibition to affect interactions between transplanted cells and regenerating axons, nor at the ability of RHO inhibitors to allow accelerated treatment of spinal cord injury during the acute care period when inflammatory responses prevent transplantation (e.g., less than or equal to 10 days after spinal cord injury, such as 1, 2, 3, 4, 5, 6, 7, 8, or 9 days after spinal cord injury). For example, even with pretreatment with RHO kinase inhibitor, bone marrow stem cells were transplanted 2 weeks after spinal cord injury (Furuya, et al. (2009) Brain Research 1295: 192-202). However, none of these references considers the in vivo effects of inhibition of RHO on cell death of endogenous cells. The effect of the Rho kinase inhibitors on endogenous cells may differ markedly from the effect on transplanted cells. For example, in Example 8 we demonstrate that the Rho kinase inhibitor Y-27632 studied by Koyanagi et al (2008) and Watanabe et al (2007) is not able to rescue neurons from trauma-induced cell death, whereas BA-210, a RHO inactivation inhibitor, has very potent neuroprotective activity. Further, none of the examples considers the ability of RHO inactivation to decrease the invasion of monocytes and decrease the immune response, nor the role of RHO inactivation to prevent cell death induced by myelin debris. Decreasing cell death in vivo by combined treatment with BA-210 will protect transplanted cells and allow transplantation within hours to days after neurotrauma or spinal cord injury. Additionally, it has not been recognized that RHO inactivation surprisingly inhibits tumor formation after cell transplantation.

Umbilical cord blood is a rich source of stem cells, including CD34+ and CD133+ stem cells (He et al. (2005) Differential gene expression profiling of CD34+CD133+ umbilical cord blood hematopoietic stem progenitor cells. Stem cells and development. 14(2):188-98). Many investigators have reported that UBCMC transplanted after SCI improve functional recovery in rats (Schira et al. (2012) Significant clinical, neuropathological and behavioral recovery from acute spinal cord trauma by transplantation of a well-defined somatic stem cell from human umbilical cord blood. Brain: a journal of neurology, 135(Pt 2):431-46; Park et al. (2012) Human umbilical cord blood-derived mesenchymal stem cell therapy promotes functional recovery of contused rat spinal cord through enhancement of endogenous cell proliferation and oligogenesis. Journal of biomedicine & biotechnology, 2012:362473. PMCID: 3304690; Kaner et al. (2010) The effects of human umbilical cord blood transplantation in rats with experimentally induced spinal cord injury. J Neurosurg Spine, 13(4):543-51; Chua et al. (2010) The effect of umbilical cord blood cells on outcomes after experimental traumatic spinal cord injury. Spine (Phila Pa. 1976), 35(16):1520-6) and dogs (Park et al. (2011). Comparison of canine umbilical cord blood-derived mesenchymal stem cell transplantation times: Involvement of astrogliosis, inflammation, intracellular actin cytoskeleton pathways, and neurotrophin. Cell Transplant; Lee et al. (2011). Schwann cell-like remyelination following transplantation of human umbilical cord blood (hUCB)-derived mesenchymal stem cells in dogs with acute spinal cord injury. J Neurol Sci, 300(1-2):86-96; Lim et al. (2007). Transplantation of canine umbilical cord blood-derived mesenchymal stem cells in experimentally induced spinal cord injured dogs. J Vet Sci, 8(3):275-82).

A number of restorative or regenerative cell sources are contemplated for use in the methods and inclusion in the product combinations disclosed herein. Examples of cell populations that comprise stem cells include cord blood cells, cord blood stems cells, neural stem cells, olfactory ensheathing glial cells (OEGs), Schwann cells, endothelial cells, endothelial progenitor cells, embryonic stem cells, adult stem cells, blood cells (e.g., placental blood, placental blood cells, umbilical cord blood, umbilical cord blood cells, umbilical cord blood cell mononuclear cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), placental stem cells nucleated cells from placental perfusate, e.g., total nucleated cells from placental perfusate, umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, adipose derived stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, hematopoietic progenitor cells, e.g., hematopoietic progenitor cells from bone marrow, fetal blood, umbilical cord blood, placental blood, and/or peripheral blood, hematopoietic stem cells contained within unprocessed placental blood, umbilical cord blood or peripheral blood; in total nucleated cells from placental blood, umbilical cord blood or peripheral blood; in an isolated population of CD34+ cells from placental blood, umbilical cord blood or peripheral blood; in unprocessed bone marrow; in total nucleated cells from bone marrow; in an isolated population of CD34+ cells from bone marrow, hematopoietic progenitor cells from bone marrow, fetal blood, umbilical cord blood, placental blood, and/or peripheral blood, Induced pluripotent stem cells (iPSCs), Bone marrow stromal stem cells, stem cells derived from brain, bone marrow, peripheral blood, blood vessels, skeletal muscle, skin, teeth, heart, gut, liver, ovarian epithelium, and testis, hematopoietic stem cells, mesenchymal stem cells, epithelial stem cells, neural stem cells, cultured stem cells, subcultured stem cells, uncultured stem cells, autochthonous stem cells, autologous stem cells, or heterologous stem cells, for example. Regenerative cells may be CD34+, CD133+, or may have other attributes. Restorative or regenerative cells may be pure or in a mixture with differentiated or undifferentiated cells.

Lithium has long been used to treat manic depression. Recent studies indicate that lithium stimulates spinal cord regeneration (Yick et al. (2004) Lithium chloride reinforces the regeneration-promoting effect of chondroitinase ABC on rubrospinal neurons after spinal cord injury. Journal of Neurotrauma, 21(7):932-43), proliferation of bone marrow and neural stem cells and suppresses astrogliogenesis (Young W. (2009) Review of lithium effects on brain and blood. Cell transplantation, 18(9):951-75). Lithium can be safely given to people with chronic spinal cord injury (Wong et al. (2011). A three-month, open-label, single-arm trial evaluating the safety and pharmacokinetics of oral lithium in patients with chronic spinal cord injury. Spinal Cord, 49(1):94-8; Yang et al. (2012). Efficacy and safety of lithium carbonate treatment of chronic spinal cord injuries: a double-blind, randomized, placebo-controlled clinical trial. Spinal Cord, 50(2):141-6). Lithium enhances the beneficial effects of UCBMC transplants in rats after spinal cord contusion (Deng et al. (2010) [Lithium chloride combined with human umbilical cord blood mesenchymal stem cell transplantation for treatment of spinal cord injury in rats]. Journal of Southern Medical University (Nan fang yi ke da xue xue bao), 30(11):2436-9).

Haemaseel is an investigational fibrin sealant being developed for use in CNS surgery. Fibrin sealants and scaffolds have a number of beneficial properties for the treatment of SCI. Fibrin sealants and scaffolds serve as efficient delivery vehicles for drugs such as BA-210 or other RHO kinase inhibitors. Additionally, fibrin sealants are used by neurosurgeons to seal dural tears (Black P. (2002) Cerebrospinal fluid leaks following spinal surgery: use of fat grafts for prevention and repair. Journal of Neurosurgery: Spine, 96(2):250-2), although no current sealants are approved for CNS surgery. Haemaseel is an investigational fibrin sealant created for CNS surgery that does not contain bovine products, has been shown safe in a 150 patient clinical study, and is produced under GMP conditions.

A number of tissue scaffolds or lattices are contemplated for use in the methods and inclusion in the product combinations disclosed herein. Examples of tissue scaffolds and lattices include scaffolds and lattices comprising collagen, biodegradable polyesters, polylactic acid, polyglycolic acid, polycaprolactone, fibrin, laminin, polysaccharidic materials, like chitosan or glycosaminoglycans (GAGs) such as hyaluronic acid, possibly cross-linked with glutaraldehyde or water soluble carbodiimide, or combinations of the above, for example; decellularized tissue extracts whereby the remaining cellular remnants or extracellular matrices act as the scaffold. Tissue scaffolds or lattices may be pre-formed to have a particular structure, or may be deliverable as liquids, suspensions, or other injectable media. Tissue scaffolds or lattices such as those comprised of at least one of the materials above may contain functionalized groups to facilitate the delivery of small molecules, such as a RHO inhibitor listed above (BA-210, for example) to specific tissues.

Accordingly, the present methods and product combinations are based, in part, on the surprising discovery that co-transplantation of cord blood cells or stem cells with BA-210 and a scaffold or lattice provides clinical benefit to spinal cord injury patients by allowing cell transplantation in a time window within the first week (e.g., less than or equal to 1, 2, 3, 4, 5, 6, or 7 days) after spinal cord injury, and also providing increased safety by inhibiting tumor formation. Further, the co-delivery of BA-210 with cell-based therapy serves to prime neurons to respond to the growth factors secreted by the cell transplants, which may overcome growth inhibition. As such, the methods described herein not only permit a substantial recovery of neurological function of the treated animals or patients, but allows successful transplantation during the critical care period by multiple synergistic activities 1) increasing survival and growth of transplanted stem cells; 2) decreasing immune response and rejection of transplanted stem cells; 3) priming neurons to overcome growth inhibition and respond to the transplanted cells; and 4) inhibiting tumor formation from the transplanted cells.

A RHO inhibitor, such as BA-210, and the regenerative cell population that comprises stem cells (e.g., umbilical cord blood cell mononuclear cells (UBMCs)) may be administered to a patient by any means known in the art. To treat or improve recovery from acute or chronic spinal cord injury, it is contemplated that in some embodiments, both the cells and the RHO inhibitor are administered together, optionally in the presence of a lattice, support, or sealing agent, such as a fibrin or laminin-based tissue sealant. Delivery may be accomplished by injection or direct application, for example, during decompression surgery. Accordingly, in some embodiments, within less than or equal to or any number in between about 1, 2, 3, 4, 5, 6, 7, 8 or 9 days post spinal cord injury, after a surgeon stabilizes the spine of a patient that has suffered a spinal cord injury, a population of restorative or regenerative cells that comprises stem cells (e.g., umbilical cord blood cell mononuclear cells (UBMCs)) are injected into or onto the spinal cord (e.g., rostral and/or caudal to or at the injury site). Before closure of the surgical wound site, a RHO inhibitor (e.g., BA-210) is applied in conjunction with a fibrin and/or laminin sealant or lattice, which occludes or partially occludes the injury site and/or the site at which the cells and RHO inhibitor are applied. Optionally, the restorative or regenerative cell population (e.g., UBMCs) may be incubated or contacted with a RHO inhibitor (e.g., BA-210) prior to administration of the cells such that the restorative or regenerative cell population and the RHO inhibitor are provided simultaneously. In these aspects, the fibrin and/or laminin sealant, or other scaffold or lattice, for example, may then be applied to the wound. Still further, the restorative or regenerative cell population (UBMC) may be provided in a fibrin and/or laminin scaffold or lattice, which may also optionally comprise a RHO inhibitor (e.g., BA-210). By this approach, the restorative or regenerative cell population, the RHO inhibitor, and the lattice or scaffold may be provided in a kit or product combination, which allows for rapid application in the clinical setting.

The RHO inhibitor (e.g., BA-210) may be provided to the patient that has experienced spinal cord injury and/or neurotrauma in less than or equal to or any number in between about 1, 2, 3, 4, 5, 6, 7, 8, 9, 10, 11, 12, 13, 14, 15, 16, 17, 18, 19, or 20 days prior to administering the restorative or regenerative cell population that comprises stem cells (e.g., UBMCs). Preferably, the RHO inhibitor (e.g., BA-210) is provided to the patient in less than or equal to or any number in between about 1, 2, 3, 4, 5, 6, 7, 8, 9, 10, 11, 12, 13, 14, 15, 16, 17, 18, 19, or 20 minutes after the restorative or regenerative cell population comprising stem cells (e.g., UBMCs) is provided to the patient.

The RHO inhibitor (e.g., BA-210) may be applied in a single dose or in a multi-dose regimen. The RHO inhibitor (e.g., BA-210) may be applied in multiple doses that are less than or equal to or any number in between about 12 hours, 24 hours, 36 hours, 48 hours, 60 hours, 72 hours, 4 days, 5 days, 6 days, or 7 days apart.

In some embodiments, the RHO inhibitor is BA-210 and the amount of BA-210 provided to the patient is less than, equal to, or any number in between about 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg or more than 9.0 mg.

The above-mentioned methods may be, optionally, supplemented by providing the patient having spinal cord injury and/or neurotrauma with a molecule comprising Lithium, such as Lithium acetate. The Lithium containing composition (e.g., Lithium acetate) may be provided to the patient at less than, equal to, or any number in between about 1, 2, 3, 4, 5, 6, 7, 8, 9, 10, 11, 12, 13, 14, 15, 16, 17, 18, 19, or 20 days prior to providing the population of restorative or regenerative cells comprising stem cells (e.g., UBMC), which may be provided to the patient in conjunction with a RHO inhibitor (e.g., BA-210). In some embodiments, the Lithium containing composition (e.g., Lithium acetate) is provided to the patient having spinal cord injury and/or neurotrauma on the same day, optionally in the same mixture, that the cell population comprising restorative or regenerative cells (e.g., UBMC) is provided. Again, the Lithium containing composition (e.g., Lithium acetate) can be provided to the patient having spinal cord injury and/or neurotrauma on the same day, optionally in the same mixture, that the cell population comprising restorative or regenerative cells (e.g., UBMC) is provided and the product combination and method of use can also include providing a RHO inhibitor (e.g., BA-210). That is, in some embodiments, the patient having spinal cord injury and/or neurotrauma is provided (a) Lithium acetate, (b) a cell population comprising restorative or regenerative cells (UBMC), and (c) a RHO inhibitor (e.g., BA-210) simultaneously or components (a)-(c) are provided to the patient in less than or equal to or any number in between about 1, 2, 3, 4, 5, 6, 7, 8, 9, 10, 11, 12, 13, 14, 15, 16, 17, 18, 19, or 20 minutes of one another. Additionally, a fibrin and/or laminin sealant can be provided to the patient in these embodiments.

The product combination can be delivered and/or the methods described herein can be performed on a patient having spinal cord injury and/or neurotrauma, for example, during decompression surgery. For example, a product combination as described herein may be delivered to a patient in less than or equal to or any number in between about 1, 2, 3, 4, 5, 6, 7, 8, 9, 10, 11, 12, 13, 14, 15, 16, 17, 18, 19, or 20 days after the patent has suffered a spinal cord injury and/or neurotrauma. In some embodiments, the product combination is delivered to a patient having spinal cord injury and/or neurotrauma when TNF-α levels are greater than 50 pg/mL, 100 pg/mL, 150 pg/mL, 200 pg/mL, 300 pg/mL, 400 pg/mL, 500 pg/mL, 600 pg/mL, 700 pg/mL, 800 pg/mL, 900 pg/mL, 1000 pg/mL, 1100 pg/mL, 1200 pg/mL, 1300 pg/mL, 1400 pg/mL, or 1500 pg/mL, indicative of inflammation in chronic SCI compared to uninjured controls. See, e.g., Hayes, K. C. et al. 2002. “Elevated serum titers of proinflammatory cytokines and CNS autoantibodies in patients with chronic spinal cord injury,” J. Neurotrauma 19(6): 653-761. TNF levels are much higher after acute SCI. In sham controls TNF levels are <5 pg/mg protein but after SCI the levels increase to >1500 pg/mg protein within 3 hours (Pearse et al. 2004. cAMP and Schwann cells promote axonal growth and functional recovery after spinal cord injury. Nature Medicine 10: 610-616). Other measures of inflammation are Interleukins (IL) such as L-2, IL-4, and IL-6 (Hyashi et al, Sequential mRNA Expression for immediate early genes, cytokines, and neurotrophins in spinal cord injury. J. Neurotrauma 17(3) 203-218). Similarly, a product combination as described herein may be delivered to a patient having spinal cord injury and/or neurotrauma when the levels of a cytokine associated with inflammation (e.g., IL-6) is at a range indicative of inflammation. In some embodiments, a range of TNF-α or IL-6 indicative of inflammation is an amount of the marker (e.g., TNF-α and/or IL-6) that is above the amount of said marker(s) present in a healthy person (e.g., a person that has not suffered spinal cord injury and/or neurotrauma).

A product combination described herein may be delivered to a patient having spinal cord injury and/or neurotrauma prior to the restoration of reduced or normal or healthy TNF-α and/or IL-6 levels. A product combination described herein may be delivered to an SCI site at a time after SCI which, according to methods in use prior to the present disclosure, a person having ordinary skill in the art would predict that an inflammation response would be toxic to any transplanted stem cell population. The product combination or the method performed may delivered as part of a first surgical intervention following an acute SCI. In a preferred embodiment, the product is provided in less than 10 days of occurrence of SCI in an identified patient. In a preferred embodiment, the product is provided in less than 5 days of occurrence of SCI in an identified patient. In a preferred embodiment, the product is provided at a time after occurrence of SCI in an identified patient at which an inflammatory response at the SCI site is not expected to have completed.

The inflammatory status of the patient having spinal cord injury and/or neurotrauma may be measured, for example, by measuring TNF-α and/or IL-6 levels at the site of SCI using conventional assays (e.g., by ELISA or by measuring monocytes with ED-1 or CD68 levels at the site of SCI).

In additional embodiments, a patient having spinal cord injury and/or neurotrauma is provided a nucleic acid (SEQ ID NO:2, for example) comprising a polynucleotide sequence encoding a RHO inhibitor (e.g., BA-210) in conjunction with a population of restorative or regenerative cells comprising stem cells (e.g., UBMC) and, optionally, a fibrin and/or laminin lattice and, optionally a composition comprising Lithium (e.g., Lithium acetate). In some of these embodiments, the population of restorative or regenerative cells comprising stem cells (e.g., UBMC) are transfected with the nucleic acid comprising a polynucleotide sequence encoding a RHO inhibitor (e.g., BA-210). In other embodiments, the nucleic acid comprising a polynucleotide sequence encoding a RHO inhibitor (e.g., BA-210) is provided to the patient by intramuscular injection at a site near the spinal cord injury and/or neurotrauma with or without electroporation so that the nucleic acid is taken up by the cells and the RHO inhibitor is expressed.

The population of cells comprising stem cells (e.g., UBMC) and the RHO inhibitor (e.g., BA-210) can be formulated for delivery to humans in many ways, such as, for example, aqueous and non-aqueous, and isotonic sterile injection solutions. These formulations can also contain antioxidants, buffers, bacteriostatics or antibiotics, and solutes that render the formulation isotonic with the blood of the intended recipient, and aqueous and non-aqueous sterile suspensions that can include suspending agents, solubilizers, thickening agents, stabilizers, and preservatives.

The population of cells comprising stem cells (e.g., UBMC) and the RHO inhibitor (e.g., BA-210) may be simultaneously applied or separately applied. The population of cells comprising stem cells (e.g., UBMC) and the RHO inhibitor (e.g., BA-210) may individually or separately be formulated as constituents of a scaffold or framework such as one comprising fibrin, laminin or both or a Haemaseel product. The scaffold or framework may be biodegradable.

Injection solutions and suspensions can be prepared from sterile powders, granules, and tablets. The dose administered to a patient, in the context of some embodiments should be sufficient to effect a beneficial therapeutic response in the patient over time. With regard to cultured cells, the dose will be determined by the efficacy of the particular cells employed and the condition of the patient, as well as the body weight or surface area of the patient to be treated. The size of the dose will also be determined by the existence, nature, and extent of any adverse side-effects that accompany the administration of a particular cell type in a particular patient. With regard to BA-210, the dose will be determined by the efficacy of BA-210 to reverse abnormal activation of RHO, which can be determined by methods known in the art, such as those described by Lord-Fontaine et al. (Lord-Fontaine, et al. (2008) J Neurotrauma 25: 11: 1309-22) and also those determined by clinical studies (Fehlings et al. (2011) J Neurotrauma 28: 787-796).

In determining the effective amount of the cultured cells to be administered in the treatment of a disease or injury described herein, one may evaluate cell toxicity, transplantation reactions, progression of the disease, and the production of anti-cell antibodies and anti-drug antibodies. For administration, the cultured cells of some embodiments can be administered in an amount effective to diminish or relieve one or more symptoms associated with the disease or injury, taking into account the side-effects of the cell type at various concentrations, as applied to the mass and overall health of the patient. Administration can be accomplished via single or divided doses.

The population of cells comprising stem cells (e.g., UBMC) and the RHO inhibitor (e.g., BA-210) can be administered to a patient alone or in conjunction with other therapeutic agents. In certain instances, the additional therapeutic agents can include chemotherapy and/or radiation therapy. In certain other instances, the additional therapeutic agents can comprise at least one growth factor such as, for example, GM-CSF, G-CSF, M-CSF, IL-3, IL-7, EPO, TPO, IL-5, VEGF, HGF, or any of the other growth factors and mediators known to be beneficial to neurotrauma and patients with neurodegenerative disorders, such as GDNF, BDNT, NT3, NGF, FGF, EGF, inosine, and others known in the art. These agents may be administered simultaneously or sequentially with the population of cells comprising stem cells (e.g., UBMC) and the RHO inhibitor (e.g., BA-210).

### EXAMPLES

The present embodiments will be described in greater detail by way of the following examples. The following examples are offered for illustrative purposes, and are not intended to limit the present embodiments in any manner. Those of skill in the art will readily recognize a variety of noncritical parameters which can be changed or modified to yield essentially the same results.

### Example 1

### Demonstration that BA-210 Attenuates the Immune Response after Spinal Cord Injury

ED-1 immunoreactivity is used to measure the number of monocytes that invade an injured spinal cord in studies of rat contusion SCI. To determine whether BA-210 affects the infiltration of hematogenous macrophages after CNS injury, we studied contusion injury in rat spinal cord tissue. Female Sprague-Dawley rats weighing 180-200 g were subjected to contusive injury of the thoracic spinal cord using New York University MASCIS impactor under isoflurane anesthesia. After a laminectomy at T7, a 10 g weight was dropped onto the dura mater from a height of 50 mm Immediately after spinal cord injury, 50 μg of BA-210 were added in admixture with Tisseel VH fibrin sealant (ImmunoAG, Vienna Austria) to the region of spinal cord injury. Control animals were injured and treated with PBS mixed in fibrin sealant. The incision was closed by suturing the muscle and fascia and the skin was closed with staples. At 48 h following spinal cord injury, rats were anesthetized and sacrificed by transcardial perfusion with 4% paraformaldehyde and a 5 mm epicenter segment of injured spinal cord were removed from perfused animals, and immersed in 4% paraformaldehyde for one hour. They were cryoprotected in 30% sucrose overnight, imbedded in OTC mounting media, and frozen over liquid nitrogen. Transverse cryostat sections were taken at a thickness of 16 um, mounted sequentially on gelatinized glass slides, and stored at −80° C. Sections were fixed with 4% paraformaldehyde for 15 min. After rinsing with PBS and incubation in blocking reagent (0.5% Triton X-100, 3% BSA, 5% normal gout serum in PBS) for 1 hr at room temperature, sections were placed into the ED-1 antibody (Chemicon), diluted 1:250 in blocking reagent, and incubated overnight at 4° C. Sections were then rinsed with PBS three times, followed by incubation in secondary antibody of goat-anti-mouse conjugated to FITC, diluted 1:250 in blocking reagent, for 1 h at room temperature. Cell counts were averaged from five sections 400 micrometers apart for each of four animals per group. The distribution patterns of ED-1 positive cells were examined in treated and control rats. At 3 days post-injury, the BA-210 treated animals had significantly reduced the number of ED-1 positive cells by 45% in gray matter and 50% in white matter (FIG. 1).

### Example 2

### The Effect of Combined Myelin and Inflammation on Cell Survival and Ability of BA-210 to Improve Cell Survival in the Presence of Tumor Necrosis Factor (TNF-α)

(a) Survival of PC-12 cells plated on poly-L-lysine (PLL) or myelin (Myelin) substrates. Bars represent mean+/−S.E.M., asterisk p<0.05. Apoptosis was induced by treatment with TNF-α and controls cultures (Ctrl) were left untreated. Combined myelin and TNF-α treatment induces cell death. BA-210 significantly increases cell survival in the presence of TNF-α and myelin.

### Example 3

### BA-210 is Cytostatic for Glioblastoma Cells

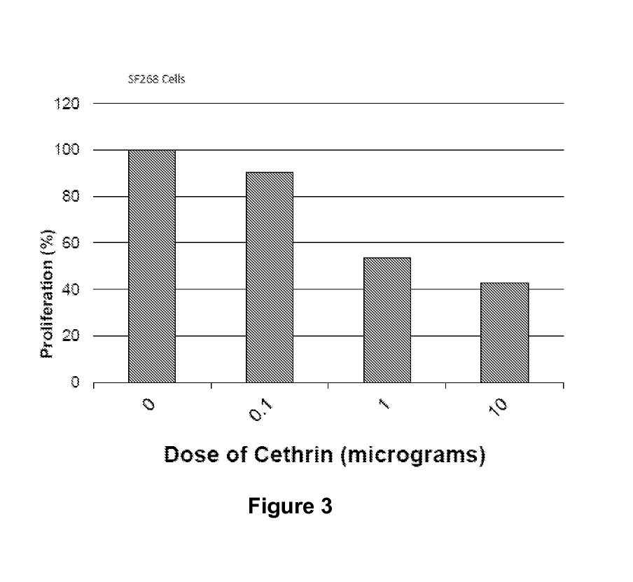

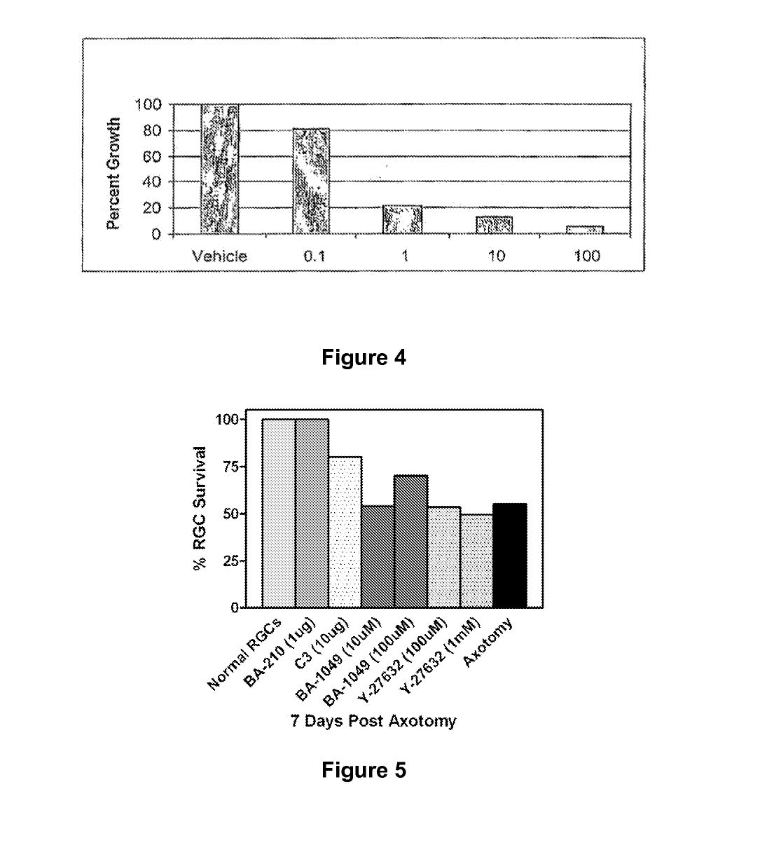

Current available models of gliomas include in vitro cell lines derived from human malignant gliomas such as the SF-268 cell line. The SF-268 glioma cell line was grown RPMI 1640 cell culture media according to manufacturer's instructions and pH adjusted to 7.10+/−0.10. A cell suspension of 20,000 cells/mL was prepared and cells were added to 8 well-chamber slides previously coated with 10 μg/mL of poly-L-lysine. After treatment with various concentrations of BA-210 for 72 hours, cell proliferation was assessed by a Sulforhodamine B (SRB) protein staining assay for the in vitro measurement of cellular protein content. This assay was developed and subsequently adopted for routine use in the National Cancer Institute for in vitro antitumor screening. The SRB binds to basic amino acids of cellular protein, and colorimetric evaluation provides an estimate of total protein mass which is related to cell number. This assay is based on the assumption that dead cells either lyse and are removed during the procedure, or otherwise do not contribute to the colorimetric end point. The assay serves as a good index of cell proliferation, and the results from plates with treated cells are compared to non-treated controls. Results of the SRB assay for SF-268 cells treated with BA-210 are shown in FIG. 3. BA-210 at doses of 1-10 mg/mL was effective to reduce the proliferation of SF-268 cells.

### Example 4

### C3-07 Treatment is Cytostatic for Glioblastoma Cells

The inhibition of growth of SF-286 CNS cancer cells by a composition of this invention comprising a fusion protein, C3-07, as measured by a sulforhodamine B (SRB) growth inhibition assay is shown. The fusion protein, C3-07, a protein related to C3-transferase, is used at concentrations of 0.1 μg/ml, 1 μg/ml, 10 μg/ml, and 100 μg/ml. At all concentrations used, cancer cell proliferation is reduced. Reduction of cancer cell proliferation is dose dependent.

### Example 5

### BA-210 Treatment and Cord Blood Cell Transplantation in Spinal Cord Injured Rats

The NYU Impactor is a device that is designed to deliver reproducible spinal cord contusions in rats. It precisely measures the movement of a 10-gram rod dropped from a specific height onto the area of the spinal cord exposed by laminectomy. Adult pathogen-free male and female Sprague-Dawley rats (8 weeks) are used. Rats are injured by contusion for behavioral studies because it best models human SCI lesions. Under isoflurane anesthesia (3-5%), rats were subjected to laminectomy at the level of T9. SCI was induced by dropping a 10-g weight rod from 25 mm height onto the exposed spinal cord using a NYU contusion impactor. The spinal cord contusion, cord blood cells (approx. 106 cells) are injected into the lesion site. BA-210 in a fibrin matrix is applied to the spinal cord after injection of the cells, as described for BA-210 treatment alone (Lord-Fontaine et al, (2008). J. Neurotrauma 25:1309-1322.). A single bolus dose of BA-210 (10 μg in 4 μL) or vehicle (PBS) is administered on the exposed cord in fibrin sealant by mixing 15 μL of thrombin with 15 μL of fibrinogen (Tisseel® kit VH, Baxter Corporation, Ontario). Postoperative treatments include 0.9% saline subcutaneously for rehydration, Buprenex for pain control and Baytril to prevent infection. Motor function of animals can be assessed using the Basso, Beattie, and Bresnahan (BBB) Open Field Locomotor Rating Scale. For the morphological evaluation of injured spinal cord, rats from the behavioral study groups are sacrificed with an overdose of anesthetic and were intracardially perfused with 0.9% saline, followed by phosphate-buffered 4% paraformaldehyde (PFA). The spinal cord tissue centered at T9 is removed from the spinal column and post-fixed in 4% PFA overnight. Ten segments of 1 mm spinal cord, in both rostral and caudal to the injury, are embedded in paraffin blocks and transversely sectioned on a microtome for use in spared tissue area measurements. The spared area of gray matter, white matter and whole sectional area of spinal cord are measured using three 5 μm thick transverse sections per level. Multiple levels were sampled at 1 mm intervals along a 2 cm region centered around the epicenter. The images are analyzed with standard image analysis software to quantitate the amount of spared tissue. To determine survival of transplanted cells, immunocytochemistry is used. For transplant survival determination, 1 cm of spinal cord (at the epicenter) is post-fixed in 4% PFA and transferred into a 30% sucrose solution. The next day, tissues are snapped-frozen in cold isopentane and embedded into optimal cutting temperature medium (OCT), and 10 μm sections placed onto glass slides and post-fixed in 4% PFA. After incubation for 1 hr in a blocking solution (5% normal goat serum, 3% BSA in PBS), BA-210 is detected using a BA-210-specific monoclonal antibody. Human cord blood cells can be detected by a polyclonal antibody to human cells, such as to specific HLA types. Double immunofluorescence is used to show that the transplanted cells take up the BA-210. It is expected that BA-210/fibrinogen treated subjects show an improvement on at least one of the post-intervention tests of neural function.

### Example 6

### BA-210 is Cytostatic for Tumor Cells Injected into CNS Tissue

A current model to test compounds for efficacy on treating glioma is to transplant cell lines of glioma cells into immune compromised mice, such as Nude mice. Studies in various tumor models have demonstrated that the specific microenvironment plays a determining role in tumor growth and selection, and the transplantation site is therefore of importance. For gliomas, literature clearly demonstrates that intracranial growth better simulates the clinical situation than subcutaneous transplantation. Similarly, for therapeutic cell transplantation, the type of injury or disease and the tissue environment of the transplanted cell will influence the tumorigenic potential of pluripotent cells and there is always some risk that cells transplanted to replace injured or diseased tissue will proliferate and form tumors.

The American type culture collection has a collection of cell lines for neurobiology. They originated from various species (human, rat, mouse). Among the human cell lines, the U-87 MG cells are widely used for in vitro and in vivo studies of human glioblastoma, and the cell line is quite resistant cell line to cytotoxic agents. To test BA-210's ability to be cytostatic to tumor cell lines tested in vivo, U-87 MG cells are maintained in Eagle MEM with 2 mM L-glutamine and Earle's BSS adjusted to contain 1.5 g/L sodium bicarbonate, 0.1 mM non-essential amino acid, and 1.0 mM sodium pyruvate, 10% fetal bovine serum. The medium is changed 2 to 3 times per week. For injection into animals, the cells are suspended, at appropriate concentration, in PBS or in cell culture medium without serum. The cells are injected into the spinal cord of 6-8 week-old female CD-1 nude mice. To expose the spinal cord for injection, the mice are anesthetized by intraperitoneal injection (i.p.) of Hypnorm and Diazepam. The fur is shaved and Proviodine solution is applied to the shaved skin for sterilization. A laminectomy is performed at the T7 vertebra, and a spinal cord injury made by contusion. The U-87 glioblastoma cells are injected into the site of spinal cord injury. BA-210 is applied to the site of cell spinal cord injury in Tisseel as described (Lord-Fontaine et al, IBID). The skin at the injury site is using 6.0 silk sutures for muscle and 4.0 for skin, respectively. The animals are placed on a heating pad for recuperation. Two weeks later survival of the transplanted cells is assessed in BA-210 treated mice and in controls that have transplanted cells without BA-210 treatment. The animals are perfused with saline and with paraformaldehyde and the spinal cord dissected and processed for cryostat sectioning. The human U-87 cells can be detected with antibodies specific for cells and a quantitative assessment of cell survival in BA-210-treated and control animals compared. In the untreated animals, the cells have proliferated and formed a tumor. In the BA-210 treated animals, the cells will survive but will not have formed a tumor.

### Example 7

### Effect of RHO or ROCK Inhibitors on TNF-α Mediated Cell Survival

Comparison of the effect of BA-210 and other RHO inhibitors on cell survival in the presence of TNF-α. PC12 cells are plated on poly-L-lysine or Myelin substrates and pre-treated with BA-210 (BA-210), wild-type Botulinum C-3 transferase, Y27362, Thiazovivin, GSK429286A, Fasudil HCl (HA-1077) or a vehicle, and subsequently treated with TNF-α or a vehicle. Combined myelin and TNF-α treatment induces cell death. RHO inhibitors increase survival in the presence of TNF-α and myelin. It is contemplated that BA-210 has a significantly greater effect on survival than other RHO inhibitors tested.

### Example 8

### Effect of RHO or ROCK Inhibitors on Retinal Ganglion Cell Survival

Comparison of the effect of BA-210 and other RHO inhibitors to promote the survival of retinal ganglion cells (RGCs) after axotomy in a rat model of optic nerve transection. First the retinal ganglion cells (RGCs) were retrogradely labeled from the superior colliculus. Next, the optic nerve was cut, without disrupting the ophthalmic artery in the ventral aspect of the optic nerve sheath. At one week after optic nerve injury, retinal whole mounts were prepared and the number of surviving RGCs was counted. We tested 100 uM and 1 mM Y-27632, 10 uM and 100 uM BA-1049, 10 ug C3-transferase and 1 ug BA-210 in our studies. No neuroprotective effect of Y-27632 was observed, and BA-1040 showed modest neuroprotection at the highest dose tested. BA-210 protected 100% of RGCs after axotomy, and C3-transferase protected 80% of RGC. Compared to the other treatments, BA-210 was most effective. RGC survival rates with BA-210 or with other RHO inhibitors are shown in FIG. 5.

### Example 9

### Effect of RHO or ROCK Inhibitors on TNF-α Mediated Cell Survival

A human neural stem cell line derived from umbilical cord blood (HUCB-NSC) cells is plated on poly-L-lysine or Myelin substrates. Cells are pre-treated with BA-210 (BA-210), wild-type C-3 transferase, Y27362, Thiazovivin, GSK429286A, Fasudil HCl (HA-1077) or a vehicle, and subsequently treated with TNF-α or a vehicle. Combined myelin and TNF-α treatment induces cell death. RHO inhibitors increase survival in the presence of TNF-α and myelin. It is contemplated that BA-210 has a significantly greater effect on survival than other RHO inhibitors tested.

### Example 10

### BA-210 Co-Treatment Increases Transplanted Cell Viability

Rats are pretreated with a TNF-α inducer to illicit elevated TNF-α levels comparable to those observed following spinal cord injury. A population of cells comprising restorative or regenerative cells, transformed with a vector directing the expression of GFP is then transplanted into the rats, either alone or accompanied by a localized treatment with BA-210 at the transplantation site. Transplanted subjects are euthanized at 8 weeks after transplantation and the GFP-labeled cell population at the transplantation site is assayed. It is contemplated that a greater number of GFP-transformed cells are observed in subjects that have been treated with BA-210 at transplantation.

### Example 11

### BA-210 Co-Treatment Decreases Transplanted Cell Tumor Formation

A population of cells comprising restorative or regenerative cells, transformed with a vector directing the expression of GFP as a cell line marker is transplanted into the rats, either alone or accompanied by a localized treatment with BA-210 at the transplantation site. Rats are administered follow-up BA-210 doses at one-week intervals for the following six weeks. Transplanted subjects are euthanized at 8 weeks after transplantation and the cell population at the site of transplantation is assayed for GFP expression and for the presence of the GFP expression vector. It is contemplated that a greater number of GFP-transformed cells or migratory cells are observed. Cells that migrate to ectopic sites pose a tumor risk. It is contemplated that tumor-like or pre-tumor-like growth is observed in samples taken from subjects that are not treated with BA-210 at transplantation.

### Example 12

### Delivery of Restorative or Regenerative Cells with Fibrinogen Networks Improves Recovery of Rats after SCI

Adult pathogen-free male and female Sprague-Dawley rats (8 weeks) are used. Rats are injured by contusion for behavioral studies because it best models human SCI lesions. Under isoflurane anesthesia (3-5%), rats were subjected to laminectomy at the level of T9. SCI was induced by dropping a 10-g weight rod from 25 mm height onto the exposed spinal cord using a NYU contusion impactor. Following the spinal cord contusion, cord blood cells (approx. 10⁶ cells) are applied to the spinal cord injury. BA-210 in a fibrin matrix is applied to the spinal cord to seal the injury after injection of the cells, as described for BA-210 treatment alone (Lord-Fontaine et al, (2008). J. Neurotrauma 25:1309-1322). An equivalent dose of 10 μg of BA-210 or vehicle (PBS) is infused within the fibrin matrix to the exposed cord in a fibrin scaffold by mixing 15 μL of thrombin with 15 μL of fibrinogen (Tisseel® kit VH, Baxter Corporation, Ontario). Postoperative treatments include 0.9% saline subcutaneously for rehydration, Buprenex for pain control and Baytril to prevent infection.

Motor function of animals is assessed using the Basso, Beattie, and Bresnahan (BBB) Open Field Locomotor Rating Scale. Sensory perception is assessed using the Semmes-Weinstein touch test. Autonomic bladder activity control is assayed using measurements of urinary expressions following providing rats with liquid. Axon recovery is assayed by euthanizing said rats with an overdose of anesthetic. Rats are intracardially perfused with 0.9% saline, followed by phosphate-buffered 4% paraformaldehyde (PFA). The spinal cord tissue centered at T9 is removed from the spinal column and post-fixed in 4% PFA overnight. Ten segments of 1 mm spinal cord, in both rostral and caudal to the injury, were embedded in paraffin blocks and transversely sectioned on a microtome for use in spared tissue area measurements. The spared area of gray matter, white matter and whole sectional area of spinal cord are measured using three 5 um thick transverse sections per level. Multiple levels were sampled at 1 mm intervals along a 2 cm region centered around the epicenter. The images are analyzed with standard image analysis software to quantitate the amount of spared tissue. To determine survival of transplanted cells, immunocytochemistry is used.

It is contemplated that rats treated with cord blood cells and BA-210-infused fibrin show greater performance on all of the tests listed above and demonstrate increased axon regeneration and better tissue preservation as compared to rats treated with cord blood cells and vehicle-infused fibrin.

### Example 13

### BA-210 Pre-Treatment Improves Rat SCI Recovery Following Administration of Restorative or Regenerative Cells and a Fibrinogen Scaffold at the Wound Site

SCI is induced as described above. BA-210 (2.5 ug in 1 uL) or vehicle (PBS) is injected into the wound site daily for four days following the injury. On day five following the induced injury, the lesion site is opened surgically, cord blood cells (approx. 106 cells) are injected into the lesion site, and the site is sealed with a fibrinogen scaffold. Postoperative treatments include 0.9% saline subcutaneously for rehydration, Buprenex for pain control and Baytril to prevent infection.

Motor function of animals is assessed using the Basso, Beattie, and Bresnahan (BBB) Open Field Locomotor Rating Scale. Sensory perception is assessed using the Semmes-Weinstein touch test. Autonomic bladder activity control is assayed using measurements of urinary expressions following providing rats with liquid. Axon recovery is assayed by euthanizing said rats with an overdose of anesthetic. Rats are intracardially perfused with 0.9% saline, followed by phosphate-buffered 4% paraformaldehyde (PFA). The spinal cord tissue centered at T9 is removed from the spinal column and post-fixed in 4% PFA overnight. Ten segments of 1 mm spinal cord, in both rostral and caudal to the injury, were embedded in paraffin blocks and transversely sectioned on a microtome for use in spared tissue area measurements. The spared area of gray matter, white matter and whole sectional area of spinal cord are measured using three 5 μm thick transverse sections per level. Multiple levels were sampled at 1 mm intervals along a 2 cm region centered around the epicenter. The images are analyzed with standard image analysis software to quantitate the amount of spared tissue. To determine survival of transplanted cells, immunocytochemistry is used.

It is contemplated that rats treated with BA-210 prior to introduction of cord blood cells and a fibrin scaffold show greater performance on all of the tests listed above and demonstrate increased axon regeneration as compared to rats pretreated with vehicle alone.

### Example 14

### BA-210 Pre-Treatment Improves Rat SCI Recovery Following Administration of Restorative or Regenerative Cells and a Fibrinogen Scaffold at the Wound Site

SCI is induced as described above. On day five following the induced injury, the lesion site is opened surgically, cord blood cells (approx. 106 cells) are injected into the lesion site, and the site is sealed with a fibrinogen scaffold. Postoperative treatments include 0.9% saline subcutaneously for rehydration, Buprenex for pain control and Baytril to prevent infection.

On day six following the induced injury, BA-210 (2.5 ug in 1 uL) or vehicle (PBS) is injected into the wound site daily for four days consecutively.

Motor function of animals is assessed using the Basso, Beattie, and Bresnahan (BBB) Open Field Locomotor Rating Scale. Sensory perception is assessed using the Semmes-Weinstein touch test. Autonomic bladder activity control is assayed using measurements of urinary expressions following providing rats with liquid. Axon recovery is assayed by euthanizing said rats with an overdose of anesthetic. Rats are intracardially perfused with 0.9% saline, followed by phosphate-buffered 4% paraformaldehyde (PFA). The spinal cord tissue centered at T9 is removed from the spinal column and post-fixed in 4% PFA overnight. Ten segments of 1 mm spinal cord, in both rostral and caudal to the injury, were embedded in paraffin blocks and transversely sectioned on a microtome for use in spared tissue area measurements. The spared area of gray matter, white matter and whole sectional area of spinal cord are measured using three 5 μm thick transverse sections per level. Multiple levels were sampled at 1 mm intervals along a 2 cm region centered around the epicenter. The images are analyzed with standard image analysis software to quantitate the amount of spared tissue. To determine survival of transplanted cells, immunocytochemistry is used.

It is contemplated that rats treated with BA-210 subsequent to introduction of cord blood cells and a fibrin scaffold show greater performance on all of the tests listed above and demonstrate increased axon regeneration as compared to rats pretreated with vehicle alone.

### Example 15

### Rats Suffering from a Chronic SCI Show Improved Performance on a Number of Measures of Spinal Cord Activity Following the Administration of BA-210, Restorative or Regenerative Cells and a Fibrinogen Scaffold at the Site of Injury

SCI is induced as described above. Post-induction treatments include 0.9% saline subcutaneously for rehydration, Buprenex for pain control and Baytril to prevent infection.

Rats suffering from a chronic SCI (defined for this model as SCI persisting for over one month following induction) and demonstrating hindered performance on the SCI assays discussed above are selected. The lesion site is accessed and cord blood cells (approx. 106 cells) are injected into the lesion site. The lesion is then sealed with a fibrinogen scaffold accompanied by a BA-210 treatment of 10 μg in 4 μL. Postoperative treatments include 0.9% saline subcutaneously for rehydration, Buprenex for pain control and Baytril to prevent infection.

Motor function of animals is assessed using the Basso, Beattie, and Bresnahan (BBB) Open Field Locomotor Rating Scale. Sensory perception is assessed using the Semmes-Weinstein touch test. Autonomic bladder activity control is assayed using measurements of urinary expressions following providing rats with liquid. Axon recovery is assayed by euthanizing said rats with an overdose of anesthetic. Rats are intracardially perfused with 0.9% saline, followed by phosphate-buffered 4% paraformaldehyde (PFA). The spinal cord tissue centered at T9 is removed from the spinal column and post-fixed in 4% PFA overnight. Ten segments of 1 mm spinal cord, in both rostral and caudal to the injury, were embedded in paraffin blocks and transversely sectioned on a microtome for use in spared tissue area measurements. The spared area of gray matter, white matter and whole sectional area of spinal cord are measured using three 5 μm thick transverse sections per level. Multiple levels were sampled at 1 mm intervals along a 2 cm region surrounding the epicenter. The images are analyzed with standard image analysis software to quantitate the amount of spared tissue. To determine survival of transplanted cells, immunocytochemistry is used.

It is contemplated that rats suffering from chronic SCI show greater performance on all of the tests listed above and demonstrate increased axon regeneration as compared to rats pretreated with vehicle alone.

### Example 16

### Clinical Studies of the Effect of Administration of a Product Combination of BA-210, a Scaffold and Restorative or Regenerative Cells on Patients Suffering from SCI

Patients suffering an acute SCI are identified. Thrombic and Cervical SCI are both contemplated in patient selection.

Patients are treated as soon as feasibly possible following the occurrence of SCI. Treatment is initiated independent of patient inflammatory status at the site of SCI, and may be concurrent with surgical decompression procedures undertaken within the first week after the occurrence of SCI.

HLA-matched UCBMCs are injected into the SCI site. A Fibirin sealant such as Haemaseel comprising 0.3, 1, 3, 6, or 9 mg of bioaccessable BA-210 is applied to the SCI site.

It is contemplated that a combination treatment of BA-210, UCBMCs and a fibrin sealant, administered during or before a surgical decompression surgery, will lead to increased locomotor recovery, axon regeneration, bladder control and sensory perception in a cohort of treated patients compared to an untreated patient population.

### Example 17

### Clinical Studies of the Effect of Administration of a Product Combination of BA-210, a Scaffold and Restorative or Regenerative Cells on Patients Suffering from Acute SCI

Patients suffering an acute SCI are identified. Thrombic and Cervical SCI are both contemplated in patient selection.

Patients are treated within 10 days following the occurrence of SCI. Treatment is initiated independent of patient inflammatory status at the site of SCI, and may be concurrent with surgical decompression procedures undertaken within the first week after the occurrence of SCI.

HLA-matched UCBMCs are injected into the SCI site. A Fibrin sealant such as Haemaseel comprising 0.3, 1, 3, 6, or 9 mg of bioaccessable BA-210 is applied to the SCI site.

It is contemplated that a combination treatment of BA-210, UCBMCs and a fibrin sealant, administered during or before a surgical decompression surgery, will lead to increased locomotor recovery, axon regeneration, bladder control and sensory perception in a cohort of treated patients compared to an untreated patient population.

It is further contemplated that patients suffering thoracic SCI may show significantly greater improvement on at least one of the tests comprising tests for increased locomotor recovery, axon regeneration, bladder control and sensory perception in a cohort of treated patients compared to an untreated patient population.

### Example 18

### Clinical Studies of the Effect of Administration of a Product Combination of BA-210, a Scaffold and Restorative or Regenerative Cells in Combination with Lithium Acetate on Patients Suffering from Acute SCI

Patients suffering an acute SCI are identified. Thoracic and Cervical SCI are both contemplated in patient selection.

Patients are treated as in examples I or II above, but are further administrated an oral dose of lithium acetate 1, 2, 3, 4, 5, 6, 7, 8, or 9 days prior to the treatment contemplated, on the day of the treatment contemplated, or 1, 2, 3, 4, 5, 6, 7, 8, or 9 days subsequent to the treatment contemplated.

It is contemplated that a combination treatment of BA-210, UCBMCs and a fibrin sealant, in combination with an oral lithium dose administered during or before a surgical decompression surgery, will lead to increased locomotor recovery, axon regeneration, bladder control and sensory perception in a cohort of treated patients compared to an untreated patient population.

It is further contemplated that patients suffering thoracic SCI may show significantly greater improvement on at least one of the tests comprising tests for increased locomotor recovery, axon regeneration, bladder control and sensory perception in a cohort of treated patients compared to an untreated patient population.

### Example 19

### Clinical Studies of the Effect of Administration of a Product Combination of BA-210, a Scaffold and Restorative or Regenerative Cells on Patients Suffering from Chronic SCI

Patients suffering chronic SCI are identified. Thrombic and Cervical SCI are both contemplated in patient selection.

The SCI site is surgically exposed. HLA-matched UCBMCs are injected into the SCI site. A Fibrin sealant such as Tisseel or Haemaseel comprising 0.3, 1, 3, 6, or 9 mg of bioaccessable BA-210 is applied to the SCI site.

It is contemplated that a combination treatment of BA-210, UCBMCs and a fibrin sealant will lead to increased locomotor recovery, axon regeneration, bladder control and sensory perception in a cohort of treated patients compared to an untreated patient population.

It is further contemplated that patients suffering thoracic SCI may show significantly greater improvement on at least one of the tests comprising tests for increased locomotor recovery, axon regeneration, bladder control and sensory perception in a cohort of treated patients compared to an untreated patient population.

### Example 20

### Clinical Studies of the Effect of Administration of a Product Combination of BA-210, a Scaffold and Stem Cells on Patients Suffering from Chronic SCI

Patients suffering chronic SCI are identified. Thrombic and Cervical SCI are both contemplated in patient selection.

The SCI site is surgically exposed. Neural stem cells such as HuCNS-SC cells are injected into the SCI site. A Fibrin sealant such as Tisseel or Haemaseel comprising 0.3, 1, 3, 6, or 9 mg of bioaccessable BA-210 is applied to the SCI site.

It is contemplated that a combination treatment of BA-210, HuCNS-SC cells and a fibrin sealant, administered during or before a surgical decompression surgery, will lead to increased locomotor recovery, axon regeneration, bladder control and sensory perception in a cohort of treated patients compared to an untreated patient population.

It is further contemplated that patients suffering thoracic SCI may show significantly greater improvement on at least one of the tests comprising tests for increased locomotor recovery, axon regeneration, bladder control and sensory perception in a cohort of treated patients compared to an untreated patient population.

It is to be understood that the above description is intended to be illustrative and not restrictive. Many embodiments will be apparent to those of skill in the art upon reading the above description. The scope of embodiments should, therefore, be determined not with reference to the above description, but should instead be determined with reference to the appended claims, along with the full scope of equivalents to which such claims are entitled. The disclosures of all articles and references, including patent applications, patents, PCT publications, and Genbank Accession Nos., are incorporated herein by reference for all purposes.

1. (canceled)

2-99. (canceled)

100. A composition for improving cell transplantation in a subject that has suffered spinal cord injury (SCI), comprising:
a) a cell population, which comprises endothelial cells, endothelial progenitor cells, umbilical cord blood cells, umbilical cord blood stem cells, umbilical cord blood cell mononuclear cells (UBMC), neural stem cells, olfactory ensheathing glial cells (OEGs), Schwann cells, bone marrow stem cells, stem cells derived from brain, embryonic stem cells, or iPS cells;
b) a tissue lattice or scaffold; and
c) BA-210.

101. The composition of claim 100, wherein said cell population is uncultured.

102. The composition of claim 100, wherein said lattice or scaffold comprises collagen, fibrin, or laminin, or a combination thereof.

103. A method of improving cell transplantation in a subject that has suffered spinal cord injury (SCI), said method comprising providing the composition of claim 100 to said subject.

104. The method of claim 103, wherein said composition is provided to said subject within 10 days of occurrence of said SCI.

105. The method of claim 103, wherein said composition is provided to said subject within 5 days of occurrence of said SCI.

106. The method of claim 103, wherein said SCI is a chronic injury.

107. The method of claim 103, wherein said cell population is uncultured.

108. The method of claim 103, wherein said lattice or scaffold comprises collagen, fibrin, or laminin, or a combination thereof.

109. The method of claim 103, wherein said composition is provided to said subject during decompression surgery.

110. A method of improving cell transplantation in a subject that has suffered spinal cord injury (SCI), said method comprising co-delivery of BA-210 and a cell population, which comprises endothelial cells, endothelial progenitor cells, umbilical cord blood cells, umbilical cord blood stem cells, umbilical cord blood cell mononuclear cells (UBMC), neural stem cells, olfactory ensheathing glial cells (OEGs), Schwann cells, bone marrow stem cells, stem cells derived from brain, embryonic stem cells, or iPS cells to said subject.

111. The method of claim 110, wherein said BA-210 and the cell population are provided to said subject within 5 days of occurrence of said SCI.

112. The method of claim 110, wherein said cell population is uncultured.

113. The method of claim 110, wherein said SCI is a chronic injury.

114. The method of claim 110, wherein said composition is provided to said subject within 10 days of occurrence of said SCI.

115. The method of claim 110, wherein said composition is provided to said subject during decompression surgery.
